(19) United States
(12) Patent Application Publication
(10) Pub. No.: US 2009/0234014 A1
(43) Pub. Date: Sep. 17, 2009

(54) Naphthalene-Disulfonamides Useful for the Treatment of Inflammation

(76) Inventors: Benjamin Pelcman, Solna (SE); Kristofer Olofsson, Solna (SE); Olga Habarova, Riga. (LV); Victor Andrianov, Riga (LV)

Correspondence Address: MORGAN LEWIS & BOCKIUS LLP, 1111 PENNSYLVANIA AVENUE NW, WASHINGTON, DC, 20004, US

(21) Appl. No.: 12/083,378

(22) PCT Filed: Oct. 13, 2006
(86) PCT No.: PCT/GB2006/003795

§ 371 Date: Jan. 9, 2009

Related U.S. Application Data
(60) Provisional application No. 60/725,639, filed on Oct. 13, 2005.

(51) Int. Cl. A61K 31/18; C07C 307/06

(52) U.S. Cl. 514/603; 564/83

(57) ABSTRACT

There is provided compounds of formula (I), wherein R¹, R², X², X⁴ and X⁵ to X⁸ have meanings given in the description, and pharmaceutically-acceptable salts thereof, which compounds are useful in the treatment of diseases in which inhibition of the activity of a member of the MAPEG family is desired and/or required, and particularly in the treatment of inflammation.

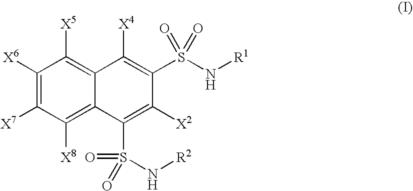

### FIELD OF THE INVENTION

This invention relates to novel pharmaceutically-useful compounds, which compounds are useful as inhibitors of enzymes belonging to the membrane-associated proteins in the eicosanoid and glutathione metabolism (MAPEG) family. Members of the MAPEG family include the microsomal prostaglandin E synthase-1 (mPGES-1), 5-lipoxygenase-activating protein (FLAP), leukotriene C₄ synthase and microsomal glutathione S-transferases (MGST1, MGST2 and MGST3). The compounds are of potential utility in the treatment of inflammatory diseases including respiratory diseases. The invention also relates to the use of such compounds as medicaments, to pharmaceutical compositions containing them, and to synthetic routes for their production.

### BACKGROUND OF THE INVENTION

There are many diseases/disorders that are inflammatory in their nature. One of the major problems associated with existing treatments of inflammatory conditions is a lack of efficacy and/or the prevalence of side effects (real or perceived).

Inflammatory diseases that affect the population include asthma, inflammatory bowel disease, rheumatoid arthritis, osteoarthritis, rhinitis, conjunctivitis and dermatitis.

Inflammation is also a common cause of pain. Inflammatory pain may arise for numerous reasons, such as infection, surgery or other trauma. Moreover, several diseases including malignancies and cardiovascular diseases are known to have inflammatory components adding to the symptomatology of the patients.

Asthma is a disease of the airways that contains elements of both inflammation and bronchoconstriction. Treatment regimens for asthma are based on the severity of the condition. Mild cases are either untreated or are only treated with inhaled β-agonists which affect the bronchoconstriction element, whereas patients with more severe asthma typically are treated regularly with inhaled corticosteroids which to a large extent are anti-inflammatory in their nature.

Another common disease of the airways with inflammatory and bronchoconstrictive components is chronic obstructive pulmonary disease (COPD). The disease is potentially lethal, and the morbidity and mortality from the condition is considerable. At present, there is no known pharmacological treatment capable of changing the course of the disease.

The cyclooxygenase (COX) enzyme exists in two forms, one that is constitutively expressed in many cells and tissues (COX-1), and one that in most cells and tissues is induced by pro-inflammatory stimuli, such as cytokines, during an inflammatory response (COX-2).

COXs metabolise arachidonic acid to the unstable intermediate prostaglandin H₂ (PGH₂). PGH₂ is further metabolized to other prostaglandins including PGE₂, PGF_{2α}, PGD₂, prostacyclin and thromboxane A₂. These arachidonic acid metabolites are known to have pronounced physiological and pathophysiological activity including pro-inflammatory effects.

PGE₂ in particular is known to be a strong pro-inflammatory mediator, and is also known to induce fever and pain. Consequently, numerous drugs have been developed with a view to inhibiting the formation of PGE₂, including “NSAIDs” (non-steroidal antiinflammatory drugs) and “coxibs” (selective COX-2 inhibitors). These drugs act predominantly by inhibition of COX-1 and/or COX-2, thereby reducing the formation of PGE₂.

However, the inhibition of COXs has the disadvantage that it results in the reduction of the formation of all metabolites downstream of PGH₂, some of which are known to have beneficial properties. In view of this, drugs which act by inhibition of COXs are therefore known/suspected to cause adverse biological effects. For example, the non-selective inhibition of COXs by NSAIDs may give rise to gastrointestinal side-effects and affect platelet and renal function. Even the selective inhibition of COX-2 by coxibs, whilst reducing such gastrointestinal side-effects, is believed to give rise to cardiovascular problems.

An alternative treatment of inflammatory diseases that does not give rise to the above-mentioned side effects would thus be of real benefit in the clinic. In particular, a drug that inhibits (preferably selectively) the transformation of PGH₂ to the pro-inflammatory mediator PGE₂ might be expected to reduce the inflammatory response in the absence of a corresponding reduction of the formation of other, beneficial arachidonic acid metabolites. Such inhibition would accordingly be expected to alleviate the undesirable side-effects mentioned above.

PGH₂ may be transformed to PGE₂ by prostaglandin E synthases (PGES). Two microsomal prostaglandin E synthases (mPGES-1 and mPGES-2), and one cytosolic prostaglandin E synthase (cPGES) have been described.

The leukotrienes (LTs) are formed from arachidonic acid by a set of enzymes distinct from those in the COX/PGES pathway. Leukotriene B₄ is known to be a strong proinflammatory mediator, while the cysteinyl-containing leukotrienes C₄, D₄ and E₄ (CysLTs) are mainly very potent bronchoconstrictors and have thus been implicated in the pathobiology of asthma. The biological activities of the CysLTs are mediated through two receptors designated CysLT₁ and CysLT₂. As an alternative to steroids, leukotriene receptor antagonists (LTRas) have been developed in the treatment of asthma. These drugs may be given orally, but do not control inflammation satisfactorily. The presently used LTRas are highly selective for CysLT₁. It may be hypothesised that better control of asthma, and possibly also COPD, may be attained if the activity of both of the CysLT receptors could be reduced. This may be achieved by developing unselective LTRas, but also by inhibiting the activity of proteins, e.g. enzymes, involved in the synthesis of the CysLTs. Among these proteins, 5-lipoxygenase, 5-lipoxygenase-activating protein (FLAP), and leukotriene C₄ synthase may be mentioned. A FLAP inhibitor would also decrease the formation of the proinflammatory LTB₄.

mPGES-1, FLAP and leukotriene C₄ synthase belong to the membrane-associated proteins in the eicosanoid and glutathione metabolism (MAPEG) family. Other members of this family include the microsomal glutathione S-transferases (MGST1, MGST2 and MGST3). For a review, c.f. P.-J. Jacobsson et al in Am. J. Respir. Crit. Care Med. 161, S20 (2000). It is well known that compounds prepared as antagonists to one of the MAPEGs may also exhibit inhibitory activity towards other family members, c.f. J. H Hutchinson et al in J. Med. Chem. 38, 4538 (1995) and D. Claveau et al in J. Immunol. 170, 4738 (2003). The former paper also describes that such compounds may also display notable cross-reactivity with proteins in the arachidonic acid cascade that do not belong to the MAPEG family, e.g. 5-lipoxygenase.

Thus, agents that are capable of inhibiting the action of mPGES-1, and thus reducing the formation of the specific arachidonic acid metabolite PGE₂, are likely to be of benefit in the treatment of inflammation. Further, agents that are capable of inhibiting the action of the proteins involved in the synthesis of the leukotrienes are also likely to be of benefit in the treatment of asthma and COPD.

### PRIOR ART

N¹,N³-Diphenyl-7-hydroxynaphthalene-1,3-disulfonamide and N¹,N³-diphenyl-7-ethoxycarbonyloxynaphthalene-1,3-disulfonamide have been disclosed in inter alia J. Pollak et al., Monatsh. Chem. 49, 203 (1928). Two chloro-substituted diphenylnaphthalene-1,3-disulfonamides have been disclosed in inter alia C. H. F. Allen, G. F. Frame, J. Org. Chem. 7, 15 (1942) and Z. Stetsura, Zh. Obshch. Khimn. 24, 2151 (1954). N¹,N³-diphenylnaphthalene-1,3-disulfonamide is disclosed in H. E. Fierz-David, C. Richter., Helv. Chim. Acta. 28, 273 (1945). Further, N¹,N³-diphenylnaphthalene-4-hydroxy-1,3-disulfonamide is disclosed in inter alia J. Pollak et al., Monatsh. Chem. 49, 187 (1928). However, none of these documents suggest the use of such compounds as pharmaceuticals.

Various 2-halonaphthalene-1,3-disulfonamides have been disclosed in inter alia JP 10088014 for use as dyes and in P. Petitcolas, R. Burel Bull. Soc. Chim. Fr., 639 (1967). Neither documents mentions or suggests the use of such compounds as pharmaceuticals.

In Complemnent and Inflammation (1991), 8, 50-59, Abdel Mawla et al disclose 5,5′,5″-(1,3,6-naphthalenetriyl-tris[sulfonylimino])-tris(1,3-benzenedisulfonic acid)hexasodium salt as a complement inhibitor, and which may therefore be useful in the treatment of inflammation. However, this document does not mention or suggest naphthalene-1,3-sulfonamides.

U.S. Pat. Nos. 4,369,191 and 4,431,638 disclose various compounds that may be useful as complement inhibitors, and thus in the treatment of inflammation. However, the former document does not mention or suggest naphthalenes that have only two sulfonamide groups attached to the naphthalene. The latter does not mention or suggest aromatic sulfonamides in which the aromatic ring is not substituted by a hexose-thio group.

### DISCLOSURE OF THE INVENTION

According to the invention there is provided a compound of formula I,

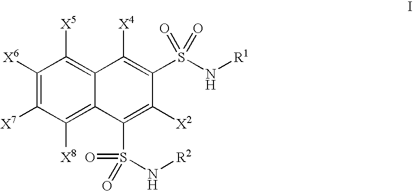

wherein
R¹ and R² independently represent aryl or heteroaryl, both of which are optionally substituted by one or more substituents selected from Z¹;
X², X⁴ and X⁵ to X⁸ independently represent hydrogen or a substituent selected from Z²;
Z¹ and Z² independently represent halo, R^{3a}, —CN, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p} or —S(O)₂N(R^{4h})R^{5h};
m represents 0, 1 or 2;
R^{3b}, R^{3d} to R^{3h}, R^{3k}, R³ⁿ, R^{4a} to R^{4h}, R^{5a}, R^{5b}, R^{5d} and R^{5f} to R^{5h} independently represent H or R^{3a}; or
any of the pairs R^{4a} and R^{5a}, R^{4b} and R^{5b}, R^{4d} and R^{5d}, R^{4f} and R^{5f}, R^{4g} and R^{5g} or R^{4h} and R^{5h} may be linked together to form a 3- to 6-membered ring, which ring optionally contains a further heteroatom (such as nitrogen or oxygen) in addition to the nitrogen atom to which these substituents are necessarily attached, and which ring is optionally substituted by F, Cl, ═O or R^{3a};
R^{3c}, R³ⁱ, R^{3j}, R^{3m} and R^{3p} independently represent R^{3a};
R^{3a} represents, on each occasion when mentioned above, C₁₋₆ alkyl optionally substituted by one or more substituents selected from F, Cl, ═O, —OR^{6a} or —N(R^{6b})R^{7b};
R^{6a} and R^{6b} independently represent H or C₁₋₆ alkyl optionally substituted by one or more substituents selected from F, Cl, ═O, —OR^{8a}, —N(R^{9a})R^{10a} or —S(O)₂-G¹;
R^{7b} represents H, —S(O)₂CH₃, —S(O)₂CF₃ or C₁₋₆ alkyl optionally substituted by one or more substituents selected from F, Cl, ═O, —OR^{11a}, —N(R^{12a})R^{13a} or —S(O)₂-G²;
or R^{6b} and R^{7b} may be linked together to form a 3- to 6-membered ring, which ring optionally contains a further heteroatom (such as nitrogen or oxygen) in addition to the nitrogen atom to which these substituents are necessarily attached, and which ring is optionally substituted by F, Cl, ═O or C₁₋₃ alkyl optionally substituted by one or more fluoro atoms;
G¹ and G² independently represent —CH₃, —CF₃ or —N(R^{14a})R^{15a};
R^{8a} and R^{11a} independently represent H, —CH₃, —CH₂CH₃ or —CF₃;
R^{9a}, R^{10a}, R^{12a}, R^{13a}, R^{14a} and R^{15a} independently represent H, —CH₃ or —CH₂CH₃,
or a pharmaceutically acceptable salt thereof,
provided that:
(A) when R¹ and R² both represent unsubstituted phenyl and X², X⁴, X⁵ and X⁸ all represent hydrogen, then:
   (I) when X⁶ represents hydrogen, X⁷ does not represent H, chloro, —OH or —OC(O)O-ethyl;
   (II) when X⁷ represents hydrogen, X⁶ does not represent chloro;
(B) when R¹ and R² both represent unsubstituted phenyl, X⁴ represents —OH, and X², X⁵, and X⁸ all represent H, then, when X⁶ represents hydrogen, X⁷ does not represent hydrogen;
(C) when X⁴, X⁵, X⁶, X⁷ and X⁸ all represent H, and R¹ and R² both represent unsubstituted phenyl or 2-methylphenyl, then X² does not represent chloro, iodo or bromo,
   which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl (where q is the upper limit of the range), defined herein may be straight-chain or, when there is a sufficient number (i.e. a minimum of three) of carbon atoms, be branched-chain, and/or cyclic (so forming, in the case of alkyl, a C_{3-q} cycloalkyl group). Further, when there is a sufficient number (i.e. a minimum of four) of carbon atoms, such groups may also be part cyclic. Further, unless otherwise specified, such alkyl groups may also be saturated or, when there is a sufficient number (i.e. a minimum of two) of carbon atoms and unless otherwise specified, be unsaturated (forming, for example, a C_{2-q} alkenyl or a C_{2-q} alkynyl group).

The term “halo”, when used herein, includes fluoro, chloro, bromo and iodo.

Aryl groups that may be mentioned include C₆₋₁₄ (e.g. C₆₋₁₀) aryl groups. Such groups may be monocyclic, bicyclic or tricyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are linked to the rest of the molecule via an atom of the aromatic ring.

Heteroaryl groups that may be mentioned include those which have between 5 and 14 (e.g. between 5 and 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heteroaryl groups that may be mentioned include acridinyl, benzimidazolyl, benzodioxanyl, benzodioxepinyl, benzodioxolyl (including 1,3-benzodioxolyl), benzofuranyl, benzofurazanyl, benzothiazolyl, benzothiadiazolyl (including 2,1,3-benzothiadiazolyl), benzoxadiazolyl (including 2,1,3-benzoxadiazolyl), benzoxazinyl (including 3,4-dihydro-2H-1,4-benzoxazinyl), benzoxazolyl, benzimidazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. However, when heteroaryl groups are bicyclic or tricyclic, they are linked to the rest of the molecule via an atom of the aromatic ring. Heteroaryl groups may also be in the N- or S-oxidised form.

Heteroatoms that may be mentioned include phosphorus, silicon, boron, tellurium, selenium and, preferably, oxygen, nitrogen and sulphur.

For the avoidance of doubt, in cases in which the identity of two or more substituents in a compound of formula I may be the same, the actual identities of the respective substituents are not in any way interdependent. For example, in the situation in which two of X⁵, X⁶, X⁷ and X⁸ represent Z², then the respective Z² groups in question may be the same or different. Similarly, when groups are substituted by more than one substituent as defined herein, the identities of those individual substituents are not to be regarded as being interdependent. For example, when R¹ represents phenyl substituted by —R^{3a} and —OR^{3h}, in which R^{3h} represents R^{3a}, and, in each case R^{3a} represents C₁₋₆ alkyl, the identities of the two R^{3a} groups are not to be regarded as being interdependent.

For the avoidance of doubt, when a term such as “X⁵ to X⁸” is employed herein, this will be understood by the skilled person to mean X⁵, X⁶, X⁷ and X⁸ inclusively.

Compounds of the invention that may be mentioned include those in which:

X² and X⁴ independently represent H;
when any of the pairs R^{4a} and R^{5a}, R^{4b} and R^{5b}, R^{4d} and R^{5d}, R^{4f} and R^{5f}, R^{4g} and R^{5g} or R^{4h} and R^{5h} are linked together, they together form a 3- to 6-membered ring, which ring optionally contains a further heteroatom (such as nitrogen or oxygen) in addition to the nitrogen atom to which these substituents are necessarily attached, and which ring is optionally substituted by ═O or R^{3a};
R^{3a} represents, on each occasion when mentioned above, C₁₋₆ alkyl optionally substituted by one or more substituents selected from F, Cl, —OCH₃, —OCH₂CH₃ or —OCF₃.

Preferred compounds of the invention include those in which:

when any of the pairs R^{4a} and R^{5a}, R^{4b} and R^{5b}, R^{4d} and R^{5d}, R^{4f} and R^{5f}, R^{4g} and R^{5g} and R^{4h} and R^{5h} are linked together, they form a 5- or 6-membered ring, which ring optionally contains a further heteroatom (such as nitrogen or oxygen) and is optionally substituted by R^{3a} (so forming, for example, a pyrrolidinyl, morpholinyl or a piperazinyl (e.g. 4-methylpiperazinyl) ring);
X² and X⁴ independently represent H or —OH;
at least one (such as at least two (e.g. three)) of X⁵ to X⁸ represent(s) hydrogen;
R¹ and R² are each, independently, substituted with less than four substituents;
Z¹ and Z² independently represent —C(O)N(R^{4a})R^{5a} or, preferably, —N(R^{4b})R^{5b}, —N(R^{3d})C(O)R^{4e} or, more preferably, halo (e.g. chloro, fluoro or bromo), —R^{3a} or —OR^{3h};
R^{3a} represents C₁₋₆ (e.g. C₁₋₄) alkyl (e.g. ethyl or, preferably, methyl) optionally substituted by one or more fluoro atoms (so forming, for example, a trifluoromethyl group);
R^{4a} and R^{5a} independently represent H;
R^{4b} and R^{5b} independently represent H, methyl or ethyl;
R^{3h} represents H or, preferably, R^{3a};
R^{4c} represents R^{3a};
when R^{3d} represents R^{3a}, then R^{3a} preferably represents C₁₋₂ alkyl (e.g. methyl);
when R^{3h} represents R^{3a}, then R^{3a} preferably represents C₁₋₆ alkyl as hereinbefore defined or more, preferably, C₁₋₃ (e.g. C₁₋₂) alkyl optionally substituted by one or more fluoro atoms (e.g. R^{3h} may represent cyclopentyl, cyclopropyl, preferably ethyl, difluoromethyl or, more preferably, methyl or trifluoromethyl);
when R^{4c} represents R^{3a}, then R^{3a} preferably represents C₁₋₆ alkyl as hereinbefore defined and preferably, unsubstituted C₁₋₆ alkyl such as cyclohexyl, cyclopropyl, tert-butyl, isopropyl, ethyl or, more preferably, methyl)
R^{6a}, R^{6b} and R^{7b} independently represent H or C₁₋₆ alkyl optionally substituted by one or more fluoro atoms.

Preferred aryl and heteroaryl groups that R¹ and R² may represent include optionally substituted phenyl, naphthyl, pyrrolyl, furanyl, thienyl (e.g. thien-2-yl or thien-3-yl), pyrazolyl, imidazolyl (e.g. 1-imidazolyl, 2-imidazolyl or 4-imidazolyl), oxazolyl, isoxazolyl, thiazolyl, pyridyl (e.g. 2-pyridyl, 3-pyridyl or 4-pyridyl), indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, group. Preferred values include pyridyl (e.g. 2- or 4-pyridyl), pyrazinyl (e.g. 2-pyrazinyl), furanyl, thienyl, oxazolyl, thiazolyl and, more preferably, phenyl.

Preferred values of Z¹ include ethyl, —C(O)NH₂ or, preferably, —OH, —CN, —N(H)C(O)CH₃, —N(CH₃)C(O)CH₃, methoxy or, more preferably, methyl, trifluoromethyl or halo (e.g. chloro, bromo or fluoro).

Preferred values of Z² include —N(H)CH₃, —N(H)S(O)₂CH₃, —N(H)S(O)₂CF₃ or, preferably, methyl, trifluoromethyl, halo (e.g. chloro, bromo or fluoro), methoxy, difluoromethoxy, trifluoromethoxy, —NH₂, —N(CH₃)₂, —CN, —N(H)C(O)CH₃, —N(CH₃)C(O)CH₃ or, more preferably, —OH.

More preferred compounds of the invention include those in which:

one of X⁵ to X⁸ (e.g. X⁶ or, more particularly, X⁷) represents H or a substituent selected from —N(H)CH₃, —N(H)S(O)₂CH₃, —N(H)S(O)₂CF₃ or, preferably, methoxy, difluoromethoxy, trifluoromethoxy, chloro, fluoro, —NH₂, —N(CH₃)₂, —N(C₂H₅)₂, —N(H)C(O)CH₃ or, more preferably, —OH, and the remaining three (e.g. X⁵, X⁷ and X⁸ or, more particularly, X⁵, X⁶ and X⁸) represent H;
Z¹ represents —OR^{3h}, —C(O)N(R^{4a})R^{5a} or, preferably, halo (e.g. chloro, fluoro or bromo) or —R^{3a};
Z² represents —OR^{3h};
when R¹ and/or R² represents phenyl substituted with three substituents, then those substituents are preferably in the 3-, 5- and 6-position, 2-, 3- and 6-position or, preferably in the 2-, 4- and 6-position;
when R¹ and/or R² represents phenyl substituted with one substituent at the 3-position, then preferred substituents include methyl or, more preferably, another Z¹ substituent as hereinbefore defined;
when R¹ and/or R² represents phenyl substituted with two substituents, then those substituents are preferably in the 3- and 5-, 3- and 4-, 2- and 4- or, particularly, 2- and 5-, 2- and 6- or, more particularly, in the 2- and 3-position.

Preferred values of R¹ and R² are:

2-pyridyl, which group is unsubstituted or substituted as hereinbefore defined (such as with one substituent (e.g. halo (e.g. bromo) or, preferably, C₁₋₃ alkyl (such as methyl)) at, for example, the 6-position or, more preferably at the 3-position);
4-pyridyl, which group is preferably unsubstituted;
2-pyrazinyl, which group is preferably unsubstituted; or, more preferably, phenyl, which group is unsubstituted or substituted as hereinbefore defined.

Preferred compounds of the invention include those in which:

X² represents H;
X⁴ represents H or, more preferably —OH;
X⁵ and/or X⁸ represent H;
X⁶ represents H;
R¹ and R² are the same.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I, which process comprises:

(i) for compounds of formula I in which R¹ and R² represent the same optionally substituted aryl or heteroaryl group, reaction of a compound of formula II,

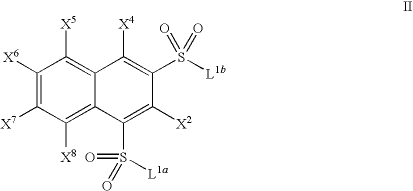

wherein L^{1a} and L^{1b} independently represent a suitable leaving group such as chloro, bromo, fluoro or —O—C₁₋₃ alkyl optionally substituted by one or more fluoro atoms (so forming for e.g. methoxy or trifluoromethoxy), and X², X⁴ and X⁵ to X⁸ are as hereinbefore defined, with a compound of formula III,

R^{x}—NH₂ III

wherein R^{x} represents both R¹ and/or R² (as appropriate), for example at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, 1,8-diazabicyclo[5.4.0]undec-7-ene, sodium hydroxide, NT-ethyldiisopropylamine, N-(methylpolystyrene)-4-(methylamino)pyridine or mixtures thereof) in an appropriate solvent (e.g. tetrahydrofuran, pyridine, toluene, dichloromethane, chloroform, acetonitrile, dimethylformamide, triethylamine, water or dimethylsulfoxide). The skilled person will appreciate that for optimum yield, at least two equivalents of a compound of formula III is required;
(ii) reaction of a compound of formula IV,

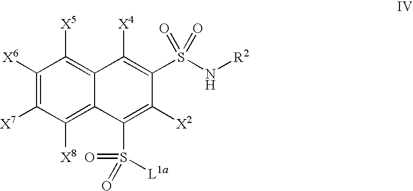

or a compound of formula V,

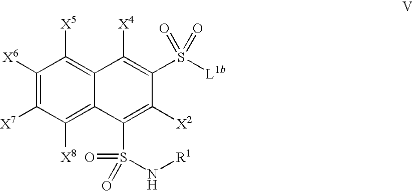

wherein X², X⁴, X⁵ to X⁸, R¹, R², L^{1a} and L^{1b} are as hereinbefore defined, with a compound of formula III as hereinbefore defined, in which R^{x} represents R¹ (for reaction with compounds of formula IV) or R² (for reaction with compounds of formula V) under standard reaction conditions, such as those described hereinbefore in respect of process step (i);
(iii) for compounds of formula I in which X⁸ represents Z², in which Z² represents halo, —R^{3a}, —C(O)R^{3b}, —C(O)OR^{3c}, —C(O)N(R^{4a})R^{5a}, —S(O)ₘR^{3j} or —S(O)₂N(R^{4h})R^{5h}, and R^{3b}, R^{4a}, R^{5a}, R^{4h} and R^{5h} are as hereinbefore defined, provided that they do not represent hydrogen, and R^{3a}, R^{3c}, and R^{3j} are as hereinbefore defined, may be synthesised by reaction of a compound corresponding to a compound of formula I but in which X⁸ represents a metal (e.g. lithium), with a compound of formula VI,

Z^{x}-L² VI

wherein L² represents a suitable leaving group, such as chloro, bromo or iodo and Z^{x} represents halo, —R^{3a}, —C(O)R^{3b}, —C(O)OR^{3c}, —C(O)N(R^{4a})R^{5a}, —S(O)ₘR^{3j} or —S(O)₂N(R^{4h})R^{5h}, and R^{3b}, R^{4a}, R^{5a}, R^{4h} and R^{5h} are as hereinbefore defined, provided that they do not represent hydrogen, and R^{3a}, R^{3c} and R^{3j} are as hereinbefore defined, under standard reaction conditions. The above-mentioned compounds corresponding to a compound of formula I but in which X⁸ represents a metal may be synthesised under standard conditions, for example by metallation (e.g. lithiation) of a corresponding compound of formula I in which X⁸ represents H, in the presence of a suitable organometallic reagent (such as an organolithuium base (e.g. n-BuLi, s-BuLi or t-BuLi)) in the presence of a suitable solvent (e.g. a polar aprotic solvent such as THF or diethyl ether), at a suitably low temperature (e.g. between −78° C. and 0° C., depending upon the strength of the organolithium base);
(iv) for compounds of formula I in which a substituent Z¹ or Z² is present and represents —N(R^{4b})R^{5b} in which R^{5b} is H and R^{4b} is as hereinbefore defined, hydrolysis of a corresponding compound of formula I in which the relevant substituent is —N(R^{4b})C(O)OR^{4c} in which R^{4b} and R^{4c} are as hereinbefore defined, or a protected derivative thereof, under standard conditions (e.g. employing aqueous acidic conditions);
(v) for compounds of formula I in which a substituent Z¹ or Z² is present and represents —C(O)OR^{3c} and/or —OC(O)OR^{3p} and R^{3c} and R^{3p} are as hereinbefore defined, trans-esterification of a corresponding compound of formula I in which R^{3c} and R^{3p} do not represent the same value as the value of R^{3c} and R^{3p} in the compound of formula I to be prepared, under standard conditions known to those skilled in the art;
(vi) for compounds of formula I in which a substituent Z¹ or Z² is present and represents —C(O)OR^{3c}, —C(O)N(R^{4a})R^{5a}, —N(R^{4b})R^{5b}, —N(R^{3e})C(O)N(R^{4d})R^{5d}, —N(R^{3f})C(O)OR^{4e}, —N(R^{3g})S(O)₂N(R^{4f})R^{5f}, —OR^{3h}, —OC(O)N(R^{4g})R^{5g}, —OC(O)OR^{3p} and/or —S(O)₂N(R^{4h})R^{5h}, and R^{3e}, R^{3f}, R^{3g}, R^{3h}, R^{4a}, R^{4b}, R^{4d}, R^{4e}, R^{4f}, R^{4g}, R^{4h}, R^{5a}, R^{5b}, R^{5d}, R^{5f}, R^{5g} and R^{5h} are as hereinbefore defined, provided that they do not represent hydrogen, and R^{3c} and R^{3p} are as hereinbefore defined, may be prepared by reaction of a compound corresponding to a compound of formula I in which R^{3c} and/or R^{3p} represents hydrogen or a corresponding compound of formula I in which R^{3e}, R^{3f}, R^{3g}, R^{3h}, R^{4a}, R^{4b}, R^{4d}, R^{4e}, R^{4f}, R^{4g}, R^{4h}, R^{5a}, R^{5b}, R^{5d}, R^{5f}, R^{5g} and/or R^{5h} represent hydrogen (as appropriate), or an appropriate anion thereof, with a compound of formula VII,

R^{3a}-L³ VII

wherein L³ represents a suitable leaving group, such as chloro, bromo, iodo or a triflate (e.g. —OS(O)₂CF₃) and R^{3a} is as hereinbefore defined, under standard conditions known to those skilled in the art, for example in the presence of a suitable base, such as one described hereinbefore in respect of process step (i). The skilled person will appreciate that in certain instances where monoalkylation is desired (or to avoid multiple alkylation generally), then the relevant group (e.g. —N(R^{4d})R^{5d}) may first need to be protected (and subsequently deprotected). In the case of reaction with an anion of a compound of formula I, e.g. a compound of formula I in which Z¹ and/or Z² represents —N(R^{3f})C(O)O⁻ or —OC(O)O⁻, the skilled person will appreciate that these derivatives may be prepared in situ from a corresponding compound of formula I in which the Z¹ and/or Z² (as appropriate) represents —N(R^{3f})H and —OH, respectively, followed by reaction in the presence of CO₂ (or a suitable source of CO₂);
(vii) for compounds of formula I in which a substituent Z¹ or Z² (e.g. Z²) is present and represents halo, —CN, —N(R^{4b})R^{5b}, —N(R^{3d})C(O)R^{4c}, —N(R^{3e})C(O)N(R^{4d})R^{5d}, —N(R^{3f})C(O)OR^{4e}, —N(R^{3g})S(O)₂N(R^{4f})R^{5f}, —OR^{3h} and/or —N(R^{3k})S(O)₂R^{3m}, and R^{3d}, R^{3e}, R^{3f}, R^{3g}, R^{3h}, R^{3k}, R^{3m}, R^{4b}, R^{4c}, R^{4d}, R^{4e}, R^{4f}, R^{5b}, R^{5d} and R^{5f} are as hereinbefore defined, reaction of a corresponding compound of formula I in which Z¹ or Z² (as appropriate) represents a suitable leaving group, such as bromo, iodo or, preferably, fluoro, chloro, nitro or a diazonium salt, with (for the introduction of a halogen group) a halogen, or an appropriate reagent that is a source of a halogen (e.g. a copper halide), or (for the introduction of the other Z¹ and/or Z² substituents mentioned above) with a compound of formula VIII,

Z^{y}-H VIII

wherein Z^{y} represents —CN, —N(R^{4b})R^{5b}, —N(R^{3d})C(O)R^{4c}, —N(R^{3e})C(O)N(R^{4d})R^{5d}, —N(R^{3f})C(O)OR^{4e}, —N(R^{3g})S(O)₂N(R^{4f})R^{5f}, —OR^{3h} or —N(R^{3k})S(O)₂R^{3m}, and R^{3d}, R^{3e}, R^{3f}, R^{3g}, R^{3h}, R^{3k}, R^{3m}, R^{4b}, R^{4c}, R^{4d}, R^{4e}, R^{4f}, R^{5b}, R^{5d} and R^{5f} are as hereinbefore defined, or a suitable derivative (e.g. salt) thereof (e.g. NaCN), under standard aromatic nucleophilic substitution conditions known to those skilled in the art. The skilled person will appreciate that diazonium salts (when employed as leaving groups) may be prepared under standard conditions known to those skilled in the art. The skilled person will also appreciate that (for example for reactions with a corresponding compound of formula I in which Z¹ or Z² (as appropriate) represents a suitable leaving group such as halo (e.g. chloro, bromo and iodo), —OSO₂CF₃, —B(OH)₂ or —Sn(R^{z})₃ (wherein R^{z} is C₁₋₆ alkyl and preferably, methyl or butyl)), the reaction may be performed in the presence of a suitable catalyst, for example a metal catalyst containing, preferably, Pd or Cu, and a base and, optionally in the presence of solvent and a ligand. Catalysts that may be mentioned include Pd₂(dba)₃ (tris(dibenzylideneacetone)dipalladium(0)), bases that may be mentioned include cesium carbonate, ligands that may be mentioned include 2,2′-bis(diphenylphosphino)-1,1′-binaphthyl and solvents that may be employed include toluene. Such reactions may be performed at elevated temperature (e.g. at about 90° C.) under an inert (e.g. argon) atmosphere.

Compounds of formula II (e.g. those in which X² or X⁴ represent —OH or X⁶ or X⁷ represents halo (e.g. chloro)) in which L^{1a} and L^{1b} each represent halo may be prepared by reaction of the corresponding naphthalene (e.g. from 2-hydroxynaphthalene, 4-hydroxynaphthalene or the 2-halonaphthalene) with a suitable reagent for the introduction of the sulfonyl halide group (e.g. halosulfonic acid), under conditions known to those skilled in the art (e.g. employing an excess of the halosulfonic acid).

Compounds of formulae II, IV and V in which L^{1a} and/or L^{1b} represents halo, such as chloro, (as appropriate) may be prepared by reaction of a corresponding compound of:

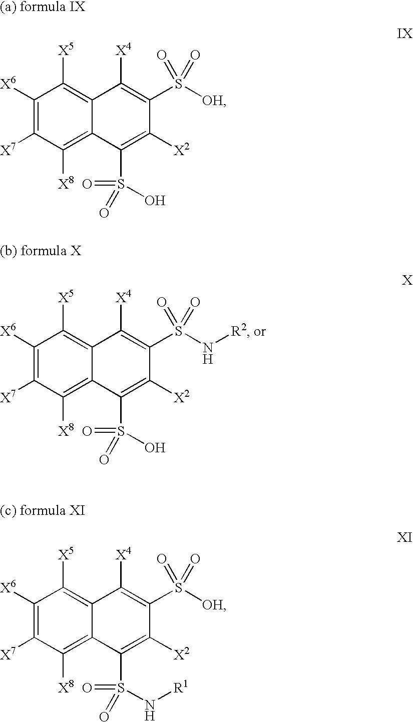

respectively, wherein X², X⁴, X⁵ to X⁸, R¹ and R² are as hereinbefore defined, with a suitable halogenating reagent, such as PCl₅, —PCl₃ or SOCl₂ (as chlorinating reagents). The skilled person will appreciate that other suitable halo groups may be prepared from the chloro derivative by an appropriate halogen exchange reaction.

Compounds of formula IV and V may alternatively be prepared by reaction of a compound of formula I with less than 2 equivalents of a compound of formula III in which R^{x} represents R¹ or R² (as appropriate), or by reaction of a compound of formula II with a mixture of two compounds of formula III, one in which R^{x} represents R¹ and the other in which R^{x} represents R², under conditions such as those hereinbefore described in respect of preparation of compounds of formula I (process step (i) above).

Compounds of formula IX (e.g. those in which X² or X⁴ represent —OH or X⁶ or X⁷ represents halo (e.g. chloro)), may be prepared by reaction of the corresponding naphthalene (e.g. 2-hydroxynaphthalene, 4-hydroxynaphthalene or the 2-halonaphthalene) with a suitable reagent for the introduction of the sulfonic acid group. Such reagents include sulphuric acid at an appropriate concentration (e.g. concentrated, fuming or H₂SO₄*H₂O), SO₃ and/or a halosulfonic acid, under conditions known to those skilled in the art.

Compounds of formulae IX, X and XI may be prepared by oxidation of a compound of:

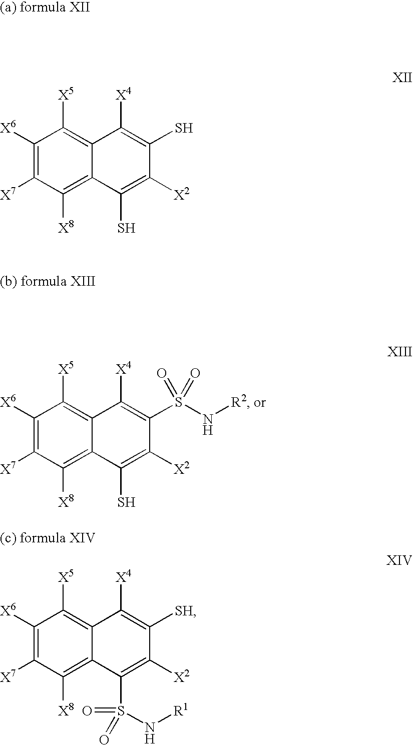

respectively, wherein X², X⁴, X⁵ to X⁸, R¹ and R² are as hereinbefore defined, under standard oxidation conditions, for example employing HNO₃ (e.g. boiling nitric acid) or m-chloroperbenzoic acid in, where necessary, an appropriate solvent system (e.g. dichloromethane).

Compounds of formula XII may be prepared by reaction of a compound of formula XV,

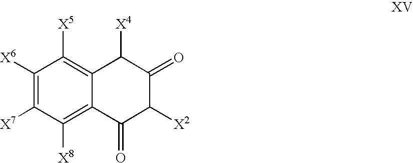

wherein X⁵ to X⁸ are as hereinbefore defined, and X² and X⁴ are as hereinbefore defined and, more preferably, H or R^{3a}, with a suitable reagent for the conversion of a carbonyl to a thiocarbonyl group (e.g. P₂S₅ or Lawesson's reagent), under conditions known to those skilled in the art.

Compounds of formula XIV may be prepared by reaction of a compound of formula XVI,

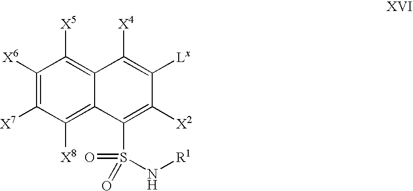

wherein L^{x} represents a suitable leaving group (such as halo (e.g. bromo)) and X², X⁴, X⁵ to X⁸ and R¹ are as hereinbefore defined, with a reagent that is a source of SH anions (e.g. NaSH), under standard conditions, for example such as those described hereinbefore in respect of preparation of compounds of formula I (process step (vii)). The skilled person will also appreciate that compounds of formula XIII may also be prepared in a similar manner from the appropriate starting material.

Compounds of formulae III, VI, VII, VIII, XIII, XV and XVI are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

The substituents X², X⁴ and X⁵ to X⁸, and optional substituents on R¹ and R², in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, and etherifications. The precursor groups can be changed to a different such group, or to the groups defined in formula I, at any time during the reaction sequence. In this respect, the skilled person may also refer to “Comprehensive Organic Functional Group Transformations” by A. R. Katritzky, O. Meth-Cohn and C. W. Rees, Pergamon Press, 1995.

Other transformations that may be mentioned include the conversion of one halo group to another, or of a halo group (preferably iodo or bromo) to a cyano or 1-alkynyl group (e.g. by reaction with a compound which is a source of cyano anions (e.g. sodium, potassium, copper (I) or zinc cyanide) or with a 1-alkyne, as appropriate). The latter reaction may be performed in the presence of a suitable coupling catalyst (e.g. a palladium and/or a copper based catalyst) and a suitable base (e.g. a tri-(C₁₋₆ alkyl)amine such as triethylamine, tributylamine or ethyldiisopropylamine). Further, amino groups and hydroxy groups may be introduced in accordance with standard conditions using reagents known to those skilled in the art.

Compounds of the invention may be isolated from their reaction mixtures using conventional techniques.

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999).

### Medical and Pharmaceutical Uses

Compounds of the invention are indicated as pharmaceuticals. According to a further aspect of the invention there is provided a compound of the invention, as hereinbefore defined but without the proviso, for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention may possess no or minimal pharmacological activity as such, but may be administered parenterally or orally, and thereafter be metabolised in the body to form compounds of the invention that possess pharmacological activity as such. Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds which possess pharmacological activity.

Compounds of the invention are particularly useful because they may inhibit the activity of a member of the MAPEG family.

Compounds of the invention are particularly useful because they may inhibit (for example selectively) the activity of prostaglandin E synthases (and particularly microsomal prostaglandin E synthase-1 (mPGES-1)), i.e. they prevent the action of mPGES-1 or a complex of which the mPGES-1 enzyme forms a part, and/or may elicit a mPGES-1 modulating effect, for example as may be demonstrated in the test described below. Compounds of the invention may thus be useful in the treatment of those conditions in which inhibition of a PGES, and particularly mPGES-1, is required.

Compounds of the invention are thus expected to be useful in the treatment of inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Accordingly, compounds of the invention may be useful in the treatment of asthma, chronic obstructive pulmonary disease, pulmonary fibrosis, inflammatory bowel disease, irritable bowel syndrome, inflammatory pain, fever, migraine, headache, low back pain, fibromyalgia, myofascial disorders, viral infections (e.g. influenza, common cold, herpes zoster, hepatitis C and AIDS), bacterial infections, fungal infections, dysmenorrhea, burns, surgical or dental procedures, malignancies (e.g. breast cancer, colon cancer, and prostate cancer), hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, neurodegenerative disorders such as Alzheimer's disease and multiple sclerosis, autoimmune diseases, allergic disorders, rhinitis, ulcers, coronary heart disease, sarcoidosis and any other disease with an inflammatory component.

Compounds of the invention may also have effects that are not linked to inflammatory mechanisms, such as in the reduction of bone loss in a subject. Conditions that may be mentioned in this regard include osteoporosis, osteoarthritis, Paget's disease and/or periodontal diseases. Compounds of the invention may thus also be useful in increasing bone mineral density, as well as the reduction in incidence and/or healing of fractures, in subjects.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, a member of the MAPEG family such as a PGES (e.g. mPGES-1), LTC₄ and/or FLAP and/or a method of treatment of a disease in which inhibition of the activity of a member of the MAPEG family such as PGES (and particularly mPGES-1), LTC₄ and/or FLAP is desired and/or required (e.g. inflammation), which method comprises administration of a therapeutically effective amount of a compound of the invention, as hereinbefore defined but without the proviso, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the life.

Such formulations may be prepared in accordance with standard and/or accepted pharmaceutical practice.

According to a further aspect of the invention there is thus provided a pharmaceutical formulation including a compound of the invention, as hereinbefore defined but without the proviso, in admixture with a pharmaceutically acceptable adjuvant, diluent or carrier.

The invention further provides a process for the preparation of a pharmaceutical formulation, as hereinbefore defined, which process comprises bringing into association a compound of the invention, as hereinbefore defined but without the proviso, or a pharmaceutically acceptable salt thereof with a pharmaceutically-acceptable adjuvant, diluent or carrier.

Compounds of the invention may also be combined with other therapeutic agents that are useful in the treatment of inflammation (e.g. NSAIDs and coxibs).

According to a further aspect of the invention, there is provided a combination product comprising:
(A) a compound of the invention, as hereinbefore defined but without the proviso; and
(B) another therapeutic agent that is useful in the treatment of inflammation,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined but without the proviso, another therapeutic agent that is useful in the treatment of inflammation, and a pharmaceutically-acceptable adjuvant, diluent or carrier; and
(2) a kit of parts comprising components:
(a) a pharmaceutical formulation including a compound of the invention, as hereinbefore defined but without the proviso, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
   which components (a) and (b) are each provided in a form that is suitable for administration in conjunction with the other.

The invention further provides a process for the preparation of a combination product as hereinbefore defined, which process comprises bringing into association a compound of the invention, as hereinbefore defined but without the proviso, or a pharmaceutically acceptable salt thereof with another therapeutic agent that is useful in the treatment of inflammation, and a pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective, and preferably selective, inhibitors of a member of MAPEG family, e.g. inhibitors of prostaglandin E synthases (PGES) and particularly microsomal prostaglandin E synthase-1 (mPGES-1). The compounds of the invention may reduce the formation of the specific arachidonic acid metabolite PGE₂ without reducing the formation of other COX generated arachidonic acid metabolites, and thus may not give rise to the associated side-effects mentioned hereinbefore.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

### Biological Test

In the assay mPGES-1 catalyses the reaction where the substrate PGH₂ is converted to PGE₂. mPGES-1 is expressed in E. coli and the membrane fraction is dissolved in 20 mM NaPi-buffer pH 8.0 and stored at −80° C. In the assay mPGES-1 is dissolved in 0.1M KPi-buffer pH 7.35 with 2.5 mM glutathione. The stop solution consists of H₂O/MeCN (7/3), containing FeCl₂ (25 mM) and HCl (0.15 M). The assay is performed at room temperature in 96-well plates. Analysis of the amount of PGE₂ is performed with reversed phase HPLC (Waters 2795 equipped with a 3.9×150 mm C18 column). The mobile phase consists of H₂O/MeCN (7/3), containing TFA (0.056%), and absorbance is measured at 195 nm with a Waters 2487 UV-detector.

The following is added chronologically to each well:
1. 100 μL mPGES-1 in KPi-buffer with glutathione. Total protein concentration: 0.02 mg/mL.
2. 1 μL inhibitor in DMSO. Incubation of the plate at room temperature for 25 minutes.
3. 4 μL of a 0.25 mM PGH₂ solution. Incubation of the plate at room temperature for 60 seconds.
4. 100 μL stop solution.
   180 μL per sample is analyzed with HPLC.

### EXAMPLES

The invention is illustrated by way of the following examples.

### Example 1

### N¹,N³-di(2,6-xylyl)-7-hydroxynaphthalene-1,3-disulfonamide

A mixture of 7-hydroxynaphthalene-1,3-disulfonyl dichloride (100 mg, 0.31 mmol), (J. Pollak, E. Blumenstock-Hulvard, Monatsh. Chem. 49, 203, (1928)), 2,6-xylidine (200 mg, 1.65 mmol) and MeCN (3 ml) was stirred at rt for 2 h. The mixture was diluted with water (30 ml) and extracted with EtOAc (30 ml). The extract was washed with HCl (aq, 4%, 30 ml), dried (Na₂SO₄) and concentrated. The residue was triturated with Et₂O to give the title compound as a colourless crystalline solid. Yield: 47 mg (31%), mp 162-164° C.

¹H NMR, (DMSO-d₆), δ: 1.85 (s, 6H), 1.92 (s, 6H), 6.90-7.11 (m, 6H), 7.32 (d, J=9.0 Hz, 1H), 7.95-8.05 (m, 1H), 8.11 (dd, J=8.7 Hz, 1H), 8.21 (d, 1H), 8.29-8.38 (m, 1H), 8.4-10.2 (br s, 3H).

### Example 2

### N¹,N³-di(o-tolyl)-7-hydroxynaphthalene-1,3-disulfonamide

The title compound was obtained in accordance with Example 1 from 7-hydroxynaphthalene-1,3-disulfonyl dichloride (100 mg, 0.29 mmol) and o-toluidine (300 mg, 2.79 mmol). Yield: 49 mg (35%) as a colourless solid, mp 118-120° C.

¹H NMR, (DMSO-d₆), δ: 1.91 (s, 3H), 1.97 (s, 3H), 6.64 (s, 1H), 6.68-6.75 (m, 1H), 6.91-7.11 (m, 8H), 7.14 (s, 1H), 7.23 (dd, J=8.8 and 2.2 Hz, 1H), 7.79 (d, J=9.0 Hz, 1H), 8.01 (d, J=2.0 Hz, 1H), 8.26 (d, J=1.6 Hz, 1H), 8.53 (d, J=1.7 Hz, 1H).

### Example 3

### N¹,N³-di(mesityl)-7-hydroxynaphthalene-1,3-disulfonamide

Pyridine (0.5 ml) was added to the solution of 7-hydroxynaphthalene-1,3-disulfonyl dichloride (100 mg, 0.29 mmol) and mesidine (100 mg, 0.74 mmol) in MeCN (2 ml). The mixture was stirred for 2 h at room temperature and concentrated. The residue was dissolved in EtOAc (30 ml) and the solution washed with HCl (aq, 4%, 30 ml), dried (Na₂SO₄) and concentrated. The residue was purified by chromatography (eluent Et₂O) to give the title compound. Yield: 52 mg (33%) as a colourless crystalline solid, mp 148-150° C.

¹H NMR, (DMSO-d₆), δ: 1.79 (s, 6H), 1.86 (s, 6H), 2.16 (s, 6H), 6.77 (d, J=4.8 Hz, 4H), 7.26-7.35 (m, 1H), 8.03 (s, 1H), 8.09 (dd, J=9.0 Hz, 1H), 8.16 (d, J=1.8 Hz, 1H), 8.30 (s, 1H), 8.6-10.4 (br s, 3H).

### Example 4

### N¹,N³-di(4-chloro-2-trifluoromethylphenyl-7-hydroxynaphthalene-1,3-disulfonamide

The title compound was obtained in accordance with Example 1 from 7-hydroxynaphthalene-1,3-disulfonyl dichloride (100 mg, 0.29 mmol), 4-chloro-2-trifluoromethylphenylamine (400 mg, 2.05 mmol) and MeCN (3 ml) after purification by chromatography (eluent Et₂O). Yield: 27 mg (14%) as a yellow solid, mp 96-98° C.

¹H NMR, (DMSO-d₆), δ: 6.98 (d, J=8.6 Hz, 1H), 7.05 (d, J=9.0 Hz, 1H), 7.36 (dd, J=9.0 and 2.2 Hz, 1H), 7.53 (d, J=8.6 Hz, 1H), 7.61 (dd, J=8.7 and 2.2 Hz, 1H), 7.69-7.79 (m, 2H), 7.95 (d, 1H), 8.19 (d, J=9.0 Hz, 1H), 8.29 (d, 1H), 8.52 (s, 1H), 10.2-10.4 (br s, 1H), 10.4-10.7 (br s, 1H), 10.73 (s, 1H).

### Example 5

### N¹,N³-di(o-anisyl)-7-hydroxynaphthalene-1,3-disulfonamide

The title compound was obtained in accordance with Example 4 from 7-hydroxynaphthalene-1,3-disulfonyl dichloride (100 mg, 0.29 mmol) and o-anisidin (0.3 ml, 2.66 mmol). Yield: 39 mg (26%) as a colourless solid, mp 99-101° C.

¹H NMR, (DMSO-d₆), δ: 3.32 (s, 3H), 3.54 (s, 3H), 5.8-6.2 (br s, 1H), 6.57 (dd, J=8.2 Hz, 1H), 6.65 (dd, J=8.2 Hz, 1H), 6.77-6.91 (m, 2H), 6.93-7.07 (m, 3H), 7.16-7.23 (m, 1H), 7.23-7.30 (m, 1H), 7.39 (dd, J=7.8 and 1.6 Hz, 1H), 7.45 (dd, J=7.8 and 1.6 Hz, 1H), 7.77 (d, J=8.9 Hz, 1H), 7.95 (d, 1H), 8.26 (d, 1H), 8.42 (d, J=1.8 Hz, 1H).

### Example 6

### N¹,N³-di(3-chloro-2-methylphenyl)-7-hydroxynaphthalene-1,3-disulfonamide

The title compound was obtained in accordance with Example 4 from 7-hydroxynaphthalene-1,3-disulfonyl dichloride (100 mg, 0.29 mmol) and 3-chloro-2-methylaniline (0.3 ml, 2.51 mmol). Yield: 39 mg (22%) as a yellow solid, mp 140-142° C.

¹H NMR, (DMSO-d₆), δ: 1.99 (s, 3H), 2.03 (s, 3H), 6.53-6.57 (m, 1H), 6.82 (dd, J=8.0 Hz, 1H), 6.98 (d, J=7.5 Hz, 1H), 7.05 (d, J=7.5 Hz, 1H), 7.22-7.32 (m, 2H), 7.34 (dd, J=9.0 and 2.2 Hz, 1H), 7.94 (d, 1H), 8.13 (d, J=9.0 Hz, 1H), 8.23 (d, J=1.8 Hz, 1H), 8.33 (d, 1H), 10.01 (s, 1H), 10.27 (s, 1H), 10.74 (s, 1H).

### Example 7

### N¹,N³-di(5-chloro-2-methylphenyl)-7-hydroxynaphthalene-1,3-disulfonamide

The title compound was obtained in accordance with Example 4 from 7-hydroxynaphthalene-1,3-disulfonyl dichloride (100 mg, 0.29 mmol) and 5-chloro-2-methylaniline (0.3 ml, 2.51 mmol). Yield: 39 mg (22%) as a yellow solid, mp 140-142° C.

¹H NMR, (DMSO-d₆), δ: 1.79 (s, 3H), 1.81 (s, 3H), 6.95-7.17 (m, 6H), 7.34 (dd, J=9.0 and 2.2 Hz, 1H), 7.96 (d, 1H), 8.15 (d, J=8.8 Hz, 1H), 8.23 (d, J=1.8 Hz, 1H), 8.40 (d, 1H), 10.02 (s, 1H), 10.23 (s, 1H), 10.75 (s, 1H).

### Example 8

### N¹,N³-di(2-chloro-6-methylphenyl)-7-hydroxynaphthalene-1,3-disulfonamide

The title compound was obtained in accordance with Example 4 from 7-hydroxynaphthalene-1,3-disulfonyl dichloride (100 mg, 0.29 mmol) and 2-chloro-6-methylaniline (0.3 ml, 2.44 mmol). Yield: 27 mg (17%) as a yellow solid, mp 139-141° C.

¹H NMR, (DMSO-d₆), δ: 2.09 (s, 3H), 2.16 (s, 3H), 5.29 (s, 1H), 7.14-7.24 (m, 5H), 7.32 (d, J=9.0 Hz, 1H), 7.96 (s, 1H), 8.12 (d, J=9.0 Hz, 1H), 8.24 (d, 1H), 8.38 (s, 1H), 9.4-10.3 (br s, 2H), 10.3-10.8 (br s, 1H).

### Example 9

### N¹,N³-di(2-trifluoromethylphenyl)-7-hydroxynaphthalene-1,3-disulfonamide

The title compound was obtained in accordance with Example 4 from 7-hydroxynaphthalene-1,3-disulfonyl dichloride (100 mg, 0.29 mmol) and 2-trifluoromethylphenylamine (0.3 mL, 2.38 mmol). Yield: 27 mg (14%) as a colourless solid, mp 80-82° C.

¹H NMR, (DMSO-d₆), δ: 5.35 (s, 1H), 6.80-7.09 (m, 2H), 7.28-7.55 (m, 4H), 7.62-7.77 (m, 2H), 8.00 (br s, 1H), 8.13-8.25 (m 1H), 8.34 (s, 1H), 8.52 (s, 1H), 10.18 (s, 1H), 10.39 (s, 1H), 10.69 (s, 1H).

### Example 10

### N¹,N³-di(2-bromophenyl)-7-hydroxynaphthalene-1,3-disulfonamide

The title compound was obtained in accordance with Example 1 from 7-hydroxynaphthalene-1,3-disulfonyl dichloride (100 mg, 0.29 mmol) and 2-bromoaniline (0.3 ml, 2.44 mmol) after purification by chromatography (eluent EtOAc:petroleum ether, 2:3) and precipitation from an etheral solution by addition of petroleum ether. Yield: 51 mg (28%) as a yellow solid, mp 154-156° C.

¹H NMR, (DMSO-d₆), δ: 7.04 (dd, J=7.8 and 1.9 Hz, 1H), 7.07-7.18 (m, 3H), 7.18-7.37 (m, 3H), 7.46-7.57 (m, 2H), 7.95 (d, 1H), 8.11 (d, J=9.0 Hz, 1H), 8.28 (d, J=1.7 Hz, 1H), 8.40 (d, 1H), 10.12 (s, 1H), 10.28 (s, 1H), 10.65 (s, 1H).

### Example 11

### N¹,N³-di(2,6-xylyl)naphthalene-1,3-disulfonamide

The title compound was obtained in accordance with Example 1 from naphthalene-1,3-disulfonyl dichloride (150 mg, 0.46 mmol) (Fierz-David H. E., Helv. Chim. Acta, 28, 257 (1945)), 2,6-xylidine (0.45 ml, 3.57 mmol) and MeCN (3 ml) after purification by chromatography (eluent EtOAc:petroleum ether, 1:3). Yield: 49 mg (22%) as a colourless solid, mp 244-246° C.

¹H NMR, (DMSO-d₆), δ: 1.83 (s, 6H), 1.91 (s, 6H), 6.94-7.15 (m, 6H), 7.72-7.92 (m, 2H), 8.28-8.40 (m, 2H), 8.57 (s, 1H), 8.69 (d, J=8.2 Hz, 1H), 9.69 (s, 1H), 9.91 (s, 1H).

### Example 12

### N¹,N³-di(o-tolyl)naphthalene-1,3-disulfonamide

The title compound was obtained in accordance with Example 11 from naphthalene-1,3-disulfonyl dichloride (100 mg, 0.31 mmol) and o-toluidine (300 mg, 2.80 mmol). Yield: 56 mg (27%), as a yellow solid, mp 160-162° C.

¹H NMR, (DMSO-d₆), δ: 1.87 (s, 3H), 1.95 (s, 3H), 6.71 (d, J=7.6 Hz, 1H), 6.83-7.15 (m, 7H), 7.71-7.92 (m, 2H), 8.27 (d, J=7.9 Hz, 1H), 8.37 (d, J=1.6 Hz, 1H), 8.50 (d, 1H), 8.74 (d, J=8.2 Hz, 1H), 9.8-10.1 (br s, 1H), 10.1-10.3 (br s, 1H).

### Example 13

### N¹,N³-di(mesityl)naphthalene-1,3-disulfonamide

The title compound was obtained in accordance with Example 11 from naphthalene-1,3-disulfonyl dichloride (100 mg, 0.31 mmol) and mesidine (300 mg, 2.22 mmol). Yield: 55 mg (34%) as a yellowish solid, mp 260-262° C.

¹H NMR, (DMSO-d₆), δ: 1.78 (s, 6H), 1.86 (s, 6H), 2.17 (s, 6H), 6.79 (s, 4H), 7.72-7.93 (m, 2H), 8.25-8.38 (m, 1H), 8.30 (d, J=1.8 Hz, 1H), 8.55 (d, 1H), 8.71 (d, J=8.2 Hz, 1H), 9.56 (s, 1H), 9.79 (s, 1H).

### Example 14

### N¹,N³-di(o-anisyl)naphthalene-1,3-disulfonamide

The title compound was obtained in accordance with Example 11 from naphthalene-1,3-disulfonyl dichloride (150 mg, 0.46 mmol) and o-anisidin (350 mg, 2.84 mmol). Yield: 45 mg (20%) as a colourless solid, mp 201-203° C.

¹H NMR, (DMSO-d₆), δ: 2.94 (s, 3H), 3.27 (s, 3H), 6.70 (dd, J=8.2 and 1.0 Hz, 1H), 6.75-6.90 (m, 3H), 7.01-7.21 (m, 4H), 7.68-7.87 (m, 2H), 8.22 (dd, J=7.8 Hz, 1H), 8.34 (d, J=1.8 Hz, 1H), 8.48 (d, 1H), 8.73 (d, J=8.5 Hz, 1H), 9.6-10.1 (br s, 2H).

### Example 15

### N¹,N³-di(3-chloro-2-methylphenyl)naphthalene-1,3-disulfonamide

The title compound was obtained in accordance with Example 11 from naphthalene-1,3-disulfonyl dichloride (150 mg, 0.46 mmol), 3-chloro-2-methylaniline (400 mg, 2.82 mmol) and MeCN (2 ml). Yield: 49 mg (20%) as a colourless solid, mp 108-110° C.

¹H NMR, (DMSO-d₆), δ: 1.91 (s, 3H), 2.02 (s, 3H), 6.62 (d, J=7.8 Hz, 1H), 6.84 (d, J=7.8 Hz, 1H), 6.98 (d, J=8.0 Hz, 1H), 7.07 (d, J=7.4 Hz, 1H), 7.27 (d, J=3.6 Hz, 1H), 7.31 (d, J=3.4 Hz, 1H), 7.73-7.94 (m, 2H), 8.27-8.39 (m, 1H), 8.34 (d, J=2.0 Hz, 1H), 8.51 (d, J=1.4 Hz, 1H), 8.69 (d, J=8.0 Hz, 1H), 10.20 (s, 1H), 10.45 (s, 1H).

### Example 16

### N¹,N³-di(5-chloro-2-methylphenyl)naphthalene-1,3-disulfonamide

The reaction of naphthalene-1,3-disulfonyl dichloride (150 mg, 0.46 mmol) and 5-chloro-2-methylaniline (400 mg, 2.82 mmol) in accordance with Example 11 gave an oil which was dissolved in Et₂O. Precipitation by addition of petroleum ether at 0° C. gave the title compound as a yellowish solid. Yield: 60 mg (24%), mp 125-127° C.

¹H NMR, (DMSO-d₆), δ: 1.71 (s, 3H), 1.77 (s, 3H), 6.95-7.18 (m, 6H), 7.73-7.95 (m, 2H), 8.27-8.42 (m, 2H), 8.58 (s, 1H), 8.72 (d, J=8.2 Hz, 1H), 10.24 (br s, 1H), 10.40 (br s, 1H).

### Example 17

### N¹,N³-di(2-chloro-6-methylphenyl)naphthalene-1,3-disulfonamide

The title compound was obtained in accordance with Example 11 from naphthalene-1,3-disulfonyl dichloride (150 mg, 0.46 mmol) and 2-chloro-6-methylaniline (400 mg, 2.82 mmol). Yield: 39 mg (16%) as a colourless solid, mp 207-209° C.

¹H NMR, (DMSO-d₆), δ: 2.11 (s, 3H), 2.18 (s, 3H), 7.16-7.25 (m, 6H), 7.72-7.91 (m, 2H), 8.27-8.38 (m, 2H), 8.58 (d, 1H), 8.70 (d, J=8.2 Hz, 1H), 10.07 (s, 1H), 10.26 (s, 1H).

### Example 18

### N¹,N³-di(2-bromophenyl)naphthalene-1,3-disulfonamide

The title compound was obtained in accordance with Example 11 from naphthalene-1,3-disulfonyl dichloride (150 mg, 0.46 mmol) and 2-bromoaniline (450 mg, 2.82 mmol. Yield: 44 mg (16%), as a colourless solid mp 178-180° C.

¹H NMR, (DMSO-d₆), δ: 7.06 (dd, J=7.8 and 1.6 Hz, 1H), 7.08-7.21 (m, 3H), 7.21-7.33 (m, 2H), 7.45-7.55 (m, 2H), 7.71-7.88 (m, 2H), 8.28 (dd, J=7.2 Hz, 1H), 8.37 (d, J=1.8 Hz, 1H), 8.57 (d, 1H), 8.70 (d, J=8.2 Hz, 1H), 10.31 (s, 1H), 10.52 (s, 1H).

### Example 19

### N¹,N³-di(2-fluorophenyl)naphthalene-1,3-disulfonamide

The title compound was obtained in accordance with Example 11 from naphthalene-1,3-disulfonyl dichloride (150 mg, 0.46 mmol) and 2-fluoroaniline (0.3 ml, 3.10 mmol). Yield: 45 mg (21%) as a colourless solid, mp 208-210° C.

¹H NMR, (DMSO-d₆), δ: 6.96-7.26 (r, 8H), 7.72-7.94 (m, 2H), 8.29 (dd, J=8.0 Hz, 1H), 8.40 (d, J=1.9 Hz, 1H), 8.60 (d, 1H), 8.75 (d, J=8.4 Hz, 1H), 10.49 (br s, 1H), 10.68 (br s, 1H).

### Example 20

### N¹,N³-di(p-tolyl)naphthalene-1,3-disulfonamide

The title compound was obtained in accordance with Example 11 from naphthalene-1,3-disulfonyl dichloride (150 mg, 0.46 mmol), p-toluidine (300 mg, 2.76 mmol) and MeCN (5 ml). Yield: 104 mg (50%) as a colourless solid, mp 221-222° C.

¹H NMR, (DMSO-d₆), δ: 2.11 (s, 3H), 2.13 (s, 3H), 6.80-6.98 (m, 8H), 7.69-7.81 (m, 1H), 7.82-7.94 (m, 1H), 8.25 (d, J=8.2 Hz, 1H), 8.54-8.61 (m, 2H), 8.73 (d, J=8.6 Hz, 1H), 10.1-11.0 (br s, 2H).

### Example 21

### N¹,N³-di(m-tolyl)naphthalene-1,3-disulfonamide

The title compound was obtained in accordance with Example 11 from naphthalene-1,3-disulfonyl dichloride (150 mg, 0.46 mmol), m-toluidine (300 mg, 2.76 mmol) and MeCN (10 ml). Yield: 129 mg (60%) as a colourless solid, mp 813-184° C.

¹H NMR, (DMSO-d₆), δ: 2.10 (s, 3H), 2.13 (s, 3H), 6.70-6.86 (m, 5H), 6.90 (s, 1H), 6.92-7.02 (m, 2H), 7.71-7.82 (m, 1H), 7.84-7.95 (r, 1H), 8.29 (d, J=8.2 Hz, 1H), 8.61 (d, J=1.8 Hz, 1H), 8.66 (d, 1H), 8.73 (d, J=8.6 Hz, 1H), 10.58 (br s, 1H), 10.86 (br s, 1H).

### Example 22

### N¹,N³-Di(3,5-dichlorophenyl)-7-hydroxynaphthalene-1,3-disulfonamide

A mixture of 7-hydroxynaphthalene-1,3-disulfonyl dichloride (100 mg, 0.29 mmol) and 3,5-dichloroaniline (400 mg, 2.5 mmol) in acetonitrile (3 mL) was stirred for 3 h at room temperature. The reaction mixture was diluted with water (20 mL) and extracted with ethyl acetate (30 mL). The extract was washed with 4% HCl (30 ml), dried with Na₂SO₄, evaporated and purified by chromatography (petroleum ether-ethylacetate, 2:1). Yield 62 mg (36%), as a yellowish powder, mp 122-124° C.

¹H NMR (DMSO-d₆), δ: 6.97 (d, J=1.7 Hz, 2H); 7.07 (d, J=1.7 Hz, 2H); 7.15 (t, 1H); 7.21 (t, J=1.7 Hz, 1H); 7.39 (dd, J=9.0, 1.9 Hz, 1H); 7.91 (d, 1H); 8.25 (d, J=9.0 Hz, 1H); 8.44 (d, J=1.8 Hz, 1H); 8.73 (d, 1H); 10.96 (s, 1H); 11.1-11.3 (br s, 1H); 11.3-11.5 (br s, 1H).

### Example 23

### N¹,N³-Di(3-chlorophenyl)-7-hydroxynaphthalene-1,3-disulfonamide

The title compound was prepared in accordance with Example 22 from 7-hydroxynaphthalene-1,3-disulfonyl dichloride (150 mg, 0.44 mmol) and 3-chloroaniline (600 mg, 4.7 mmol). Yield 57 mg (25%), as a yellowish powder, mp 206-208° C.

¹H NMR (DMSO-d₆), δ: 6.90-7.25 (m, 8H); 7.35 (dd, J=9.0, 2.1 Hz, 1H); 7.92 (d, 1H); 8.17 (d, J=9.0 Hz, 1H); 8.46 (d, J=1.8 Hz, 1H); 8.58 (d, 1H); 10.78-10.90 (br s, 2H); 11.0-11.15 (br s, 1H).

### Example 24

### N¹,N³-Di(2,3-dichlorophenyl)-7-hydroxynaphthalene-1,3-disulfonamide

The title compound was prepared in accordance with Example 22 from 7-hydroxynaphthalene-1,3-disulfonyl dichloride (150 mg, 0.44 mmol) and 2,3-dichloroaniline (600 mg, 3.7 mmol). Yield 55 mg (21%), as a white powder, mp 117-119° C.

¹H NMR (DMSO-d₆), δ: 7.09 (dd, J=8.1, 1.5 Hz, 1H); 7.13-7.29 (m, 3H); 7.33 (dd, J=9.0, 2.1 Hz, 1H); 7.38-7.46 (m, 1H); 7.47 (dd, J=8.1, 1.5 Hz, 1H); 7.92 (d, 1H); 8.13 (d, J=9.0 Hz, 1H); 8.33 (d, J=1.9 Hz, 1H); 8.45 (d, 1H); 10.45 (s, 1H); 10.63 (s, 1H); 10.72 (s, 1H).

### Example 25

### N¹,N³-Di(3,4-dichlorophenyl)-7-hydroxynaphthalene-1,3-disulfonamide

The title compound was prepared in accordance with Example 22 from 7-hydroxynaphthalene-1,3-disulfonyl dichloride (100 mg, 0.29 mmol) and 3,4-dichloroaniline (400 mg, 2.5 mmol). Yield 50 mg (29%), as a white powder, mp 158-160° C.

¹H NMR (DMSO-d₆), δ: 6.99 (dd, J=8.9, 2.5 Hz, 1H); 7.06 (dd, J=8.9, 2.5 Hz, 1H); 7.18 (d, J=2.5 Hz, 1H); 7.25 (d, J=2.5 Hz, 1H); 7.34-7.41 (m, 1H); 7.35 (d, J=8.8 Hz, 1H); 7.42 (d, J=8.8 Hz, 1H); 7.90 (d, J=2.0 Hz, 1H); 8.20 (d, J=9.0 Hz, 1H); 8.45 (d, J=1.7 Hz, 1H); 8.64 (d, 1H); 10.92 (s, 1H); 10.98 (s, 1H); 11.25 (s, 1H).

### Example 26

### N¹,N³-Di(2,5-dichlorophenyl)-7-hydroxynaphthalene-1,3-disulfonamide

The title compound was prepared in accordance with Example 22 from 7-hydroxynaphthalene-1,3-disulfonyl dichloride (150 mg, 0.44 mmol) and 2,5-dichloroaniline (600 mg, 3.7 mmol). Yield 52 mg (20%), as a white powder, mp 214-216° C.

¹H NMR (DMSO-d₆), δ: 7.23-7.27 (m, 3H); 7.27-7.31 (m, 2H); 7.32 (dd, J=9.0, 2.1 Hz, 1H); 7.36 (d, J=8.5 Hz, 1H); 7.94 (s, 1H); 8.15 (d, J=9.0 Hz, 1H); 8.29 (d, J=1.6 Hz, 1H); 8.47 (s, 1H); 10.49 (s, 1H); 10.64 (s, 1H); 10.72 (s, 1H).

### Example 27

### N¹,N³-di(2-hydroxyphenyl)-7-hydroxynaphthalene-1,3-disulfonamide

The title compound was prepared in accordance with Example 22 from 7-hydroxynaphthalene-1,3-disulfonyl dichloride (150 mg, 0.44 mmol) and 2-amino-phenol (500 mg, 4.6 mmol). Yield 41 mg (19%), as a yellowish solid, mp 118-120° C.

¹H NMR (DMSO-d₆), δ: 6.53-6.74 (m, 4H); 6.80-7.04 (m, 4H); 7.28 (dd, J=9.0, 2.1 Hz, 1H); 8.00 (d, J=2.2 Hz, 1H); 8.03 (d, J=9.0 Hz, 1H); 8.36 (s, 2H); 9.1-9.8 (br s, 4H); 10.2-10.8 (br s, 1H).

### Example 28

### N¹,N³-Di(2-ethylphenyl)-7-hydroxynaphthalene-1,3-disulfonamide

The title compound was prepared in accordance with Example 22 from 7-hydroxynaphthalene-1,3-disulfonyl dichloride (150 mg, 0.44 mmol) and 2-ethylaniline (500 mg, 4.1 mmol). Yield 71 mg (32%), as a light grey powder, mp 127-129° C.

¹H NMR (DMSO-d₆), δ: 0.80 (t, J=7.5 Hz, 3H); 0.93 (t, J=7.5 Hz, 3H); 2.45 (q, J=7.5 Hz, 2H); 2.50 (q, J=7.5 Hz, 2H, overlapped with signals of DMSO); 6.59 (d, J=7.9 Hz, 1H); 6.77 (d, J=7.9 Hz, 1H); 6.87-7.03 (m, 2H); 7.06-7.23 (m, 4H); 7.34 (dd, J=8.8, 2.1 Hz, 1H); 8.01 (d, 1H); 8.12 (d, J=9.0 Hz, 1H); 8.26 (d, J=1.8 Hz, 1H); 8.35 (d, 1H); 9.75 (s, 1H); 10.00 (s, 1H); 10.68 (s, 1H).

### Example 29

### N¹,N³-Di(m-tolyl)-7-hydroxynaphthalene-1,3-disulfonamide

The title compound was prepared in accordance with Example 22 from 7-hydroxynaphthalene-1,3-disulfonyl dichloride (150 mg, 0.44 mmol) and m-toluidine (500 mg, 4.76 mmol). Yield 61 mg (29%), as a brownish solid, mp 115-117° C.

¹H NMR (DMSO-d₆), δ: 2.12 (s, 3H); 2.13 (s, 3H); 6.69-6.91 (m, 6H); 6.92-7.06 (m, 2H); 7.32 (dd, J=9.0, 2.2 Hz, 1H); 7.95 (d, J=2.1 Hz, 1H); 8.11 (d, J=9.0 Hz, 1H); 8.49 (s, 2H); 9.6-11.2 (br s, 3H).

### Example 30

### N¹,N³-Di(3-fluorophenyl)-7-hydroxynaphthalene-1,3-disulfonamide

The title compound was prepared in accordance with Example 22 from 7-hydroxynaphthalene-1,3-disulfonyl dichloride (150 mg, 0.44 mmol) and 3-fluoroaniline (500 mg, 4.5 mmol). Yield 56 mg (26%), as a yellowish solid, mp 110-112° C.

¹H NMR (DMSO-d₆), δ: 6.69-6.95 (m, 6H); 7.07-7.25 (m, 2H); 7.35 (dd, J=9.0, 1.8 Hz, 1H); 7.93 (d, 1H); 8.16 (d, J=9.0 Hz, 1H); 8.48 (d, 1H); 8.58 (s, 1H); 10.4-11.4 (br s, 3H).

### Example 31

### N¹,N³-Di(3-chloro-2-methylphenyl)-7-methoxynaphthalene-1,3-disulfonamide

The title compound was prepared in accordance with Example 22 from 7-methoxynaphthalene-1,3-disulfonyl dichloride (150 mg, 0.44 mmol) and 3-chloro-2-methylaniline (300 μL, d=1.17, 2.5 mmol). The product was purified by chromatography (benzene-CHCl₃-Me₂CO, 4:16:1). Yield 33 mg (13%), as a white powder, mp 166-168° C.

¹H NMR (DMSO-d₆), δ: 1.88 (s, 3H); 2.01 (s, 3H); 3.83 (s, 3H); 6.59 (dd, J=8.0, 1.0 Hz, 1H); 6.88 (dd, J=8.0, 1.0 Hz, 1H); 6.98 (dd, J=8.0, 8.0 Hz, 1H); 7.05 (dd, J=8.0, 8.0 Hz, 1H); 7.27 (dd, J=8.0, 1.0 Hz, 1H); 7.41 (dd, J=9.2, 2.4 Hz, 1H); 7.88 (d, J=2.4 Hz, 1H); 8.19 (d, J=9.2 Hz, 1H); 8.33 (d, 1H); 8.40 (d, J=1.8 Hz, 1H); 10.06 (s, 1H); 10.43 (s, 1H).

### Example 32

### N¹,N³-di(o-tolyl)-4-hydroxynaphthalene-1,3-disulfonamide

The title compound was prepared in accordance with Example 22 from 4-hydroxynaphthalene-1,3-disulfonyl dichloride (200 mg, 0.59 mmol) and o-toluidine (600 mg, 5.7 mmol). Yield 42 mg (15%), as a yellow powder, mp 220-222° C.

¹H NMR (DMSO-d₆), δ: 1.97 (s, 3H); 2.27 (s, 3H); 6.79-7.11 (m, 8H); 7.24-7.35 (m, 1H); 7.40-7.52 (m, 1H); 7.92 (s, 1H); 8.24-8.38 (m, 2H); 8.3-8.9 (br s, 1H); 9.07 ppm (s, 1H).

### Example 33

### N¹,N³-Di(3-bromophenyl)-7-hydroxynaphthalene-1,3-disulfonamide

The title compound was prepared in accordance with Example 22 from 7-hydroxynaphthalene-1,3-disulfonyl dichloride (200 mg, 0.59 mmol) and 3-bromoaniline (600 mg, 3.5 mmol). Yield 69 mg (19%), as a yellow solid, mp 198-200° C.

¹H NMR (DMSO-d₆), δ: 6.96-7.21 (m, 7H); 7.22-7.27 (m, 1H); 7.36 (dd, J=9.0, 2.1 Hz, 1H); 7.92 (d, J=2.1 Hz, 1H); 8.17 (d, J=9.0 Hz, 1H); 8.46 (d, J=1.9 Hz, 1H); 8.59 (d, J=1.8 Hz, 1H); 10.81 (s, 1H); 10.86 (s, 1H); 11.07 (s, 1H).

### Example 34

### N¹,N³-Di(3,4,5-trichlorophenyl)-7-hydroxynaphthalene-1,3-disulfonamide

The title compound was prepared in accordance with Example 22 from 7-hydroxynaphthalene-1,3-disulfonyl dichloride (200 mg, 0.59 mmol) and 3,4,5-trichloroaniline (700 mg, 3.6 mmol). The product was purified by chromatography (eluent-ether). Yield 58 mg (15%), as a white powder, mp 164-166° C.

¹H NMR (DMSO-d₆), δ: 7.18 (s, 2H); 7.25 (s, 2H); 7.40 (dd, J=9.0, 2.2 Hz, 1H); 7.90 (d, J=2.0 Hz, 1H); 8.26 (d, J=9.0 Hz, 1H); 8.46 (d, 1H); 8.75 (d, 1H); 10.97 (s, 1H); 11.1-11.6 (br s, 2H).

### Example 35

### N¹,N³-Di(3-carbamoylphenyl)-7-hydroxynaphthalene-1,3-disulfonamide

The title compound was prepared in accordance with Example 22 from 7-hydroxynaphthalene-1,3-disulfonyl dichloride (200 mg, 0.59 mmol) and 3-aminobenzamide (600 mg, 4.41 mmol). The product was purified by chromatography (eluent-ether). Yield 50 mg (16%), as a yellow powder, mp 169-171° C.

¹H NMR (DMSO-d₆), δ: 7.03-7.50 (m, 9H); 7.52 (s, 1H); 7.65 (s, 1H); 7.80-7.99 (m, 3H); 8.11 (d, J=9.0 Hz, 1H); 8.43-8.50 (m, 2H); 10.64 (s, 1H); 10.79 (s, 1H); 10.92 (s, 1H).

### Example 36

### N¹,N³-Di(3-chloro-2-methylphenyl)-4-hydroxynaphthalene-1,3-disulfonamide

The title compound was prepared in accordance with Example 22 from 4-hydroxynaphthalene-1,3-disulfonyl dichloride (200 mg, 0.59 mmol) and 3-chloro-2-methylaniline (600 mg, 4.2 mmol). The product was purified by chromatography (ethyl acetate). Yield 55 mg (17%), as a light yellow powder, mp 245-247° C.

¹H NMR (DMSO-d₆), δ: 2.00 (s, 3H); 2.33 (s, 3H); 6.81 (dd, J=8.0, 1.4 Hz, 1H); 6.89 (d, J=8.0 Hz, 1H); 6.92-6.99 (m, 2H); 7.08-7.18 (m, 2H); 7.27-7.38 (m, 1H); 7.41-7.53 (in 1H); 7.86 (s, 1H); 8.23 (d, J=8.4 Hz, 1H); 8.34 (dd, J=8.3, 1.3 Hz, 1H); 9.33 ppm (s, 1H).

### Example 37

### N¹,N³-Di(3-chloro-2,6-diethylphenyl)-7-hydroxynaphthalene-1,3-disulfonamide

A mixture of 7-hydroxynaphthalene-1,3-disulfonyl dichloride (255 mg, 0.75 mmol) and 2,6-diethylaniline (550 mg, 3.0 mmol) in acetonitrile (3 mL) was heated under reflux during 4 h. The reaction mixture was diluted with water (30 mL) and extracted with ethyl acetate (30 mL). The extract was washed with 4% HCl (30 mL), dried with Na₂SO₄, evaporated and purified by chromatography (petroleum ether-ethylacetate, 2:1). Yield 21 mg (4%), as a white powder, mp 142-144° C.

¹H NMR (DMSO-d₆), δ: 0.75-0.98 (m, 12H); 2.05-2.46 (r, 8H); 7.09 (dd, J=8.4, 2.1 Hz, 2H); 7.32 (d, J=8.4 Hz, 2H); 7.38 (dd, J=9.0, 2.2 Hz, 1H); 7.94 (d, J=2.2 Hz, 1H); 8.19 (d, J=9.1 Hz, 1H); 8.28 (d, J=1.9 Hz, 1H); 8.45 (d, J=1.9 Hz, 1H); 9.78 (s, 1H); 10.01 (s, 1H); 10.65 (s, 1H).

### Example 38

### N¹,N³-Di(3-chloro-2-fluorophenyl)-7-hydroxynaphthalene-1,3-disulfonamide

The title compound was prepared in accordance with Example 22 from 7-hydroxynaphthalene-1,3-disulfonyl dichloride (255 mg, 0.75 mmol) and 3-chloro-2-fluoroaniline (600 mg, 4.1 mmol). Yield 48 mg (11%), as a white solid, mp 126-128° C.

¹H NMR (DMSO-d₆), δ: 6.97-7.15 (m, 4H); 7.26-7.43 (m, 3H); 7.94 (d, J=2.2 Hz, 1H); 8.17 (d, J=9.1 Hz, 1H); 8.33 (d, J=1.9 Hz, 1H); 8.51 (d, 1H); 10.62 (s, 1H); 10.80 (s, 1H); 10.84 (s, 1H).

### Example 39

### N¹,N³-Di(3-chloro-2-hydroxyphenyl-7-hydroxynaphthalene-1,3-disulfonamide

The title compound was prepared in accordance with Example 22 from 7-hydroxynaphthalene-1,3-disulfonyl dichloride (255 mg, 0.75 mmol) and 3-chloro-2-hydroxyaniline (430 mg, 3.0 mmol). Yield 52 mg (13%), as a brownish powder, mp 135-137° C.

¹H NMR (DMSO-d₆), δ: 6.57 (dd, J=8.1, 4.0 Hz, 1H); 6.61 (dd, J=8.1, 4.0 Hz, 1H); 6.72 (dd, J=8.1, 1.6 Hz, 1H); 6.75 (dd, J=8.1, 1.6 Hz, 1H); 7.07 (dd, J=8.0, 1.6 Hz, 1H); 7.12 (dd, J=8.0, 1.7 Hz, 1H); 7.32 (dd, J=8.9, 2.3 Hz, 1H); 7.97 (d, J=2.3 Hz, 1H); 8.11 (d, J=8.9 Hz, 1H); 8.36 (d, J=1.9 Hz, 1H); 8.44 (d, J=1.9 Hz, 1H); 9.55 (br s, 2H); 9.75 (s, 1H); 9.80 (s, 1H); 10.67 (s, 1H).

### Example 40

### N¹,N³-Di(3-fluoro-2-methylphenyl)-7-hydroxynaphthalene-1,3-disulfonamide

The title compound was prepared in accordance with Example 22 from 7-hydroxynaphthalene-1,3-disulfonyl dichloride (150 mg, 0.44 mmol) and 3-fluoro-2-methylaniline (400 mg, 3.2 mmol). Yield 54 mg (24%), as a white powder, mp 128-130° C.

¹H NMR (DMSO-d₆), δ: 1.83 (d, J=2.3 Hz, 3H); 1.90 (d, J=2.3 Hz, 3H); 6.50-6.60 (m, 1H); 6.70-6.77 (m, 1H); 6.89-7.13 (m, 4H), 7.33 (dd, J=9.0, 2.2 Hz, 1H); 7.96 (d, J=2.2 Hz, 1H); 8.12 (d, J=9.0 Hz, 1H); 8.24 (d, J=1.8 Hz, 1H); 8.35 (d, 1H); 9.98 (s, 1H); 10.23 (s, 1H); 10.74 (s, 1H).

### Example 41

### N¹,N³-Di(3-bromo-2-methylphenyl)-7-hydroxynaphthalene-1,3-disulfonamide

The title compound was prepared in accordance with Example 22 from 7-hydroxynaphthalene-1,3-disulfonyl dichloride (170 mg, 0.50 mmol) and 3-bromo-2-methylaniline (500 mg, 2.7 mmol). Yield 65 mg (20%), as a white powder, mp 129-131° C.

¹H NMR (DMSO-d₆), δ: 2.05 (s, 3H); 2.08 (s, 3H); 6.67 (dd, J=8.0, 1.0 Hz, 1H); 6.85 (dd, J=8.1, 1.3 Hz, 1H); 6.95 (t, 8.0 Hz, 1H); 6.97 (t, 8.0 Hz, 1H); 7.35 (dd, J=8.9, 2.3 Hz, 1H); 7.44 (dd, J=8.0, 1.0 Hz, 1H); 7.46 (dd, J=8.0, 1.0 Hz, 1H); 7.96 (d, J=2.3 Hz, 1H); 8.15 (d, J=9.0 Hz, 1H); 8.24 (d, J=1.9 Hz, 1H); 8.34 (d, J=1.9 Hz, 1H); 10.04 (s, 1H); 10.30 (s, 1H); 10.75 (s, 1H).

### Example 42

### N¹,N³-Di(3-bromo-2-methylphenyl)-4-hydroxynaphthalene-1,3-disulfonamide

The title compound was prepared in accordance with Example 22 from 4-hydroxynaphthalene-1,3-disulfonyl dichloride (120 mg, 0.39 mmol) and 3-bromo-2-methylaniline (400 mg, 2.2 mmol). Yield 61 mg (25%), as a yellowish powder, mp 246-248° C.

¹H NMR (DMSO-d₆), δ: 2.06 (s, 3H); 2.37 (s, 3H); 3.0-3.6 (br s, 1H); 6.76-7.01 (m, 4H); 7.24-7.41 (m, 3H); 7.42-7.55 (m, 1H); 7.87 (s, 1H); 8.24 (d, J=8.3 Hz, 1H); 8.37 (dd, J=8.0 and 1.0 Hz, 1H); 9.37 (s, 2H).

### Example 43

### N¹,N³-Di(2,4-dimethylphenyl)-7-hydroxynaphthalene-1,3-disulfonamide

The title compound was prepared in accordance with Example 22 from 7-hydroxynaphthalene-1,3-disulfonyl dichloride (100 mg, 0.29 mmol) and 2,4-dimethylaniline (300 mg, 2.5 mmol). Yield 70 mg (47%), as a white powder, mp 116-118° C.

¹NMR (DMSO-d₆), δ: 1.89 (s, 3H); 1.90 (s, 3H); 2.13 (s, 3H); 2.15 (s, 3H); 6.53 (d, J=8.0 Hz, 1H); 6.61-6.79 (m, 3H); 6.88-6.89 (m, 2H); 7.31 (dd, J=8.8, 2.4 Hz, 1H); 7.98 (d, J=2.0 Hz, 1H); 8.08 (d, J=9.0 Hz, 1H); 8.19 (d, J=2.0 Hz, 1H); 8.27 (d, J=1.8 Hz, 1H); 9.59 (br s, 1H); 9.84 (br s, 1H); 10.64 (br s, 1H).

### Example 44

### N¹,N³-Di(4-chloro-2-methylphenyl)-7-hydroxynaphthalene-1,3-disulfonamide

The title compound was prepared in accordance with Example 22 from 7-hydroxynaphthalene-1,3-disulfonyl dichloride (100 mg, 0.29 mmol) and 4-chloro-2-methylaniline (400 mg, 2.8 mmol). The product was purified by chromatography (ethyl acetate-petroleum ether, 2:3), and then crystallised from benzene. Yield 90 mg (56%), as a white powder, mp 140-142° C.

¹H NMR (DMSO-d₆), δ: 1.91 (s, 3H); 1.94 (s, 3H); 6.75 (d, J=8.6 Hz, 1H); 6.89 (d, J=8.6 Hz, 1H); 7.04-7.11 (m, 1H); 7.18-7.21 (m, 2H); 7.35 (dd, J=9.0, 2.2 Hz, 1H); 7.97 (d, J=2.0 Hz, 1H); 8.14 (d, J=9.0 Hz, 1H); 8.22 (d, J=2.0 Hz, 1H); 8.36 (d, J=2.0 Hz, 1H); 9.88 (s, 1H); 10.12 (s, 1H); 10.75 (s, 1H).

### Example 45

### N¹,N³-Di(3-chloro-2-methoxyphenyl)-7-hydroxynaphthalene-1,3-disulfonamide

The title compound was prepared in accordance with Example 22 from 7-hydroxynaphthalene-1,3-disulfonyl dichloride (100 mg, 0.29 mmol) and 3-chloro-2-methoxyaniline (400 mg, 2.5 mmol). The product was purified by crystallisation from benzene. Yield 60 mg (35%), as a white powder, mp 164-166° C.

¹H NMR (DMSO-d₆), δ: 3.23 (s, 3H); 3.47 (s, 3H); 6.89 (dd, J=8.0, 8.0 Hz, 1H); 6.96 (dd, J=8.0, 8.0 Hz, 1H); 7.08 (dd, J=8.4, 1.8 Hz, 1H); 7.13-7.20 (m, 3H); 7.36 (dd, J=9.0, 2.2 Hz, 1H); 8.03 (d, J=2.0 Hz, 1H); 8.18 (d, J=9.0 Hz, 1H); 8.45 (d, J=1.8 Hz, 1H); 8.61 (d, J=1.6 Hz, 1H); 10.29 (s, 1H); 10.44 (s, 1H); 10.7-10.8 (br s, 1H).

### Example 46

### N¹,N³-Di(4-methoxy-2-methylphenyl)-7-hydroxynaphthalene-1,3-disulfonamide

The title compound was prepared in accordance with Example 22 from 7-hydroxynaphthalene-1,3-disulfonyl dichloride (100 mg, 0.29 mmol) and 4′-methoxy-2-methylaniline (400 mg, 2.9 mmol). Yield 38 mg (24%), as a white powder, mp 211-213° C.

¹H NMR (DMSO-d₆), δ: 1.91 (s, 3H); 1.94 (s, 3H); 3.64 (s, 3H); 3.66 (s, 3H); 6.47-6.74 (m, 6H); 7.33 (dd, J=9.0, 2.3 Hz, 1H); 7.98 (d, J=2.2 Hz, 1H); 8.10 (d, J=9.0 Hz, 1H); 8.17 (d, J=2.0 Hz, 1H); 8.27 (d, J=1.8 Hz, 1H); 9.52 (s, 1H); 9.77 (s, 1H); 10.66 (s, 1H).

### Example 47

### N¹,N³-Di(6-bromopyridin-2-yl)naphthalene-1,3-disulfonamide

A mixture of naphthalene-1,3-disulfonyl dichloride (976 mg, 3.0 mmol), 2-amino-6-bromopyridine (1557 mg, 9.0 mmol) and MeCN (60 mL) was heated at reflux for 100 h. The mixture was cooled to room temperature, 5% aqueous HCl (50 mL) was added and the mixture was extracted with ethyl acetate (3×25 mL). The combined extract were dried with Na₂SO₄, concentrated and 2/3 of the residue was purified by chromatography (petroleum ether-ethylacetate-MeCN, 2:1:0.27) to give 230 mg (19%) of product containing 7% 2-amino-6-bromopyridine. 132 mg of this material was crystallised from H₂O—HCl-MeOH to give 70 mg of the title product as a white powder, mp 205-208° C.

¹H NMR (DMSO-d₆), δ: 6.92 (d, J=8.1 Hz, 1H), 7.03 (d, J=8.1 Hz, 1H), 7.07 (d, J=8.1 Hz, 1H), 7.19 (d, J=7.6 Hz, 1H), 7.48 (t, J=7.9 Hz, 1H), 7.61 (t, J=7.9 Hz, 1H), 7.77-7.87 (m, 1H), 7.88-8.00 (m, 1H), 8.38 (d, J=8.1 Hz, 1H), 8.68-8.80 (m, 2H), 9.04 (d, 1H), 11.5-12.3 (br s, 2H).

### Example 48

### N¹,N³-Di(3-bromophenyl)naphthalene-1,3-disulfonamide

A mixture of naphthalene-1,3-disulfonyl dichloride (163 mg, 0.50 mmol), 3-bromoaniline (258 mg, 1.50 mmol) and MeCN (10 ml) was heated at reflux for 100 h. The mixture was left at room temperature for 4 h, washed with 5% aqueous HCl (20 mL) and extracted with ethyl acetate (3×20 mL). The combined extracts were dried with Na₂SO₄, concentrated to dryness and purified by chromatography (methylene chloride-MeOH, 40:1) then by petroleum ether-ethylacetate, 2:1) to give 30 mg (10%) of the title product as a yellowish solid, mp 183-186° C.

¹H NMR (DMSO-d₆), δ: 6.90-7.04 (m, 3H); 7.06-7.19 (m, 4H); 7.24-7.29 (m, 1H); 7.71-7.82 (m, 1H); 7.84-7.95 (m, 1H); 8.30 (d, J=8.1 Hz, 1H); 8.57 (d, J=1.8 Hz, 1H); 8.71 (d, 1H); 8.76 (d, J=8.6 Hz, 1H); 10.7-11.3 (br s, 2H).

### Example 49

The following compound was tested in the biological test described above and was found to exhibit 50% inhibition of mPGES-1 at a concentration of 10 μM or below:
N¹,N³-Diphenyl-7-hydroxynaphthalene-1,3-disulfonamide.

### Example 50

Title compounds of the Examples were tested in the biological test described above and were found to exhibit 50% inhibition of mPGES-1 at a concentration of 10 μM or below. For example, the following representative compounds of the examples exhibited the following IC₅₀ values:

### Example 3: 1100 nM

### Example 7: 1200 nM

### Example 9: 4100 nM

### Example 11: 1700 nM

### Example 15: 1700 nM

### Example 16: 1300 nM

Example 20: 3700 nM

1. A compound of formula I, 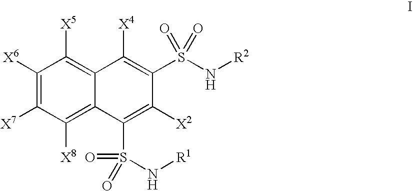
wherein
R¹ and R² independently represent aryl or heteroaryl, both of which are optionally substituted by one or more substituents selected from Z¹;
X², X⁴ and X⁵ to X⁸ independently represent hydrogen or a substituent selected from Z²;
Z¹ and Z² independently represent halo, —R^{3a}, —CN, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p} or —S(O)₂N(R^{4h})R^{5h};
m represents 0, 1 or 2;
R^{3b}, R^{3d} to R^{3h}, R^{3k}, R³ⁿ, R^{4a} to R^{4h}, R^{5a}, R^{5b}, R^{5d} and R^{5f} to R^{5h} independently represent H or R^{3a}; or
any of the pairs R^{4a} and R^{5a}, R^{4b} and R^{5b}, R^{4d} and R^{5d}, R^{4f} and R^{5f}, R^{4g} and R^{5g} or R^{4h} and R^{5h} may be linked together to form a 3- to 6-membered ring, which ring optionally contains a further heteroatom in addition to the nitrogen atom to which these substituents are necessarily attached, and which ring is optionally substituted by F, Cl, ═O or R^{3a};
R^{3c}, R³ⁱ, R^{3j}, R^{3m} and R^{3p} independently represent R^{3a};
R^{3a} represents C₁₋₆ alkyl optionally substituted by one or more substituents selected from F, Cl, ═O, —OR^{6a} or —N(R^{6b})R^{7b};
R^{6a} and R^{6b} independently represent H or C₁₋₆ alkyl optionally substituted by one or more substituents selected from F, Cl, ═O, —OR^{8a}, —N(R^{9a})R^{10a} or —S(O)₂-G¹;
R^{7b} represents H, —S(O)₂CH₃, —S(O)₂CF₃ or C₁₋₆ alkyl optionally substituted by one or more substituents selected from F, Cl, ═O, —OR^{11a}, —N(R^{12a})R^{13a} or —S(O)₂-G²; or R^{6b} and R^{7b} may be linked together to form a 3- to 6-membered ring, which ring optionally contains a further heteroatom in addition to the nitrogen atom to which these substituents are necessarily attached, and which ring is optionally substituted by F, Cl, ═O or C₁₋₃ alkyl optionally substituted by one or more fluoro atoms;
G¹ and G² independently represent —CH₃, —CF₃ or —N(R^{14a})R^{15a};
R^{8a} and R^{11a} independently represent H, —CH₃, —CH₂CH₃ or —CF₃;
R^{9a}, R^{10a}, R^{12a}, R^{13a}, R^{14a} and R^{15a} independently represent H, —CH₃ or —CH₂CH₃,
or a pharmaceutically acceptable salt thereof,
provided that:
(A) when R¹ and R² both represent unsubstituted phenyl and X², X⁴, X⁵ and X⁸ all represent hydrogen, then:
(I) when X⁶ represents hydrogen, X⁷ does not represent H, chloro, —OH or —OC(O)O-ethyl;
(II) when X⁷ represents hydrogen, X⁶ does not represent chloro;
(B) when R¹ and R² both represent unsubstituted phenyl, X⁴ represents —OH, and X², X⁵, and X⁸ all represent H, then, when X⁶ represents hydrogen, X⁷ does not represent hydrogen;
(C) when X⁴, X⁵, X⁶, X⁷ and X⁸ all represent H, and R¹ and R² both represent unsubstituted phenyl or 2-methylphenyl, then X² does not represent chloro, iodo or bromo.

2. A compound as claimed in claim 1, wherein, when any of the pairs R^{4a} and R^{5a}, R^{4b} and R^{5b}, R^{4d} and R^{5d}, R^{4f} and R^{5f}, R^{4g} and R^{5g} or R^{4h} and R^{5h} are linked together, they together form a 3- to 6-membered ring, which ring optionally contains a further heteroatom in addition to the nitrogen atom to which these substituents are necessarily attached, and which ring is optionally substituted by ═O or R^{3a}.

3. A compound as claimed in claim 1 or claim 2, wherein R^{3a} represents C₁₋₆ alkyl optionally substituted by one or more substituents selected from F, Cl, —OCH₃, —OCH₂CH₃ or —OCF₃.

4. A compound as claimed in claim 1, wherein at least two of X⁵ to X⁸ represent hydrogen.

5. A compound as claimed in claim 1, wherein R¹ and R² are each, independently, substituted with less than four substituents.

6. A compound as claimed in claim 1, wherein Z¹ and Z² independently represent —C(O)N(R^{4a})R^{5a}, —N(R^{4b})R^{5b}, —N(R^{3d})C(O)R^{4c}, halo, —R^{3a} or —OR^{3h}.

7. A compound as claimed in claim 1, wherein R^{3a} represents C₁₋₆ alkyl optionally substituted by one or more fluoro atoms.

8. A compound as claimed in claim 1, wherein R^{3h} represents R^{3a}.

9. A compound as claimed in claim 8, wherein R^{3a} represents C₁₋₃ alkyl optionally substituted by one or more fluoro atoms.

10. A compound as claimed in claim 9, wherein R^{3a} represents methyl or trifluoromethyl.

11. A compound as claimed in claim 1, wherein R¹ and R² independently represent an optionally substituted phenyl, naphthyl, pyrrolyl, furanyl, thienyl, pyrazolyl, imidazolyl, oxazolyl, isoxazolyl, thiazolyl, pyridyl, indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, or benzodioxanyl, group.

12. A compound as claimed in claim 11, wherein R¹ and R² independently represent optionally substituted phenyl, pyridyl, pyrazinyl, furanyl, thienyl, oxazolyl or thiazolyl.

13. A compound as claimed in claim 12, wherein R¹ and R² independently represent optionally substituted phenyl.

14. A compound as claimed in claim 11, wherein the optional substituents are selected from ethyl, —C(O)NH₂, —OH, —CN, —N(H)C(O)CH₃, —N(CH₃)C(O)CH₃, methoxy, methyl, trifluoromethyl and halo.

15. A compound as claimed in claim 14, wherein the optional substituents are selected from methyl, trifluoromethyl and halo.

16. A compound as claimed in claim 1, wherein Z² represents —N(H)CH₃, —N(H)S(O)₂CH₃, —N(H)S(O)₂CF₃, methyl, trifluoromethyl, halo, methoxy, difluoromethoxy, trifluoromethoxy, —NH₂, —N(CH₃)₂, —CN, —N(H)C(O)CH₃, —N(CH₃)C(O)CH₃ or —OH.

17. A compound as claimed in claim 16, wherein Z² represents —OH.

18. A compound as claimed in claim 16, wherein X² and X⁴ independently represent H or —OH.

19. A compound as claimed in claim 18, wherein X² and X⁴ independently represent H.

20. A compound as defined in claim 1, but without the proviso, or a pharmaceutically-acceptable salt thereof, for use as a pharmaceutical.

21. A pharmaceutical formulation including a compound as defined in claim 1, but without the proviso, or a pharmaceutically-acceptable salt thereof, in admixture with a pharmaceutically acceptable adjuvant, diluent or carrier.

22.-24. (canceled)

25. A method as claimed in claim 27, wherein the disease is inflammation.

26. A method as claimed in claim 27 wherein the disease is asthma, chronic obstructive pulmonary disease, pulmonary fibrosis, inflammatory bowel disease, irritable bowel syndrome, inflammatory pain, fever, migraine, headache, low back pain, fibromyalgia, a myofascial disorder, a viral infection, a bacterial infection, a fungal infection, dysmenorrhea, a burn, a surgical or dental procedure, a malignancy, hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, a neurodegenerative disorder, an autoimmune disease, an allergic disorder, rhinitis, an ulcer, coronary heart disease, sarcoidosis, any other disease with an inflammatory component, osteoporosis, osteoarthritis, Paget's disease or a periodontal disease.

27. A method of treatment of a disease in which inhibition of the activity of a member of the MAPEG family is desired and/or required, which method comprises administration of a therapeutically effective amount of a compound as defined in claim 1, but without the proviso, or a pharmaceutically-acceptable salt thereof, to a patient suffering from, or susceptible to, such a condition.

28. A method as claimed in claim 27, wherein the member of the MAPEG family is microsomal prostaglandin E synthase-1, leukotriene C₄ and/or 5-lipoxygenase-activating protein.

29. A method as claimed in claim 28, wherein the member of the MAPEG family is microsomal prostaglandin E synthase-1.

30. A combination product comprising:
(A) a compound as defined in claim 1, but without the proviso, or a pharmaceutically-acceptable salt thereof; and
(B) another therapeutic agent that is useful in the treatment of inflammation,
wherein each of components (A) and (B) is formulated in admixture with a pharmaceutically-acceptable adjuvant, diluent or carrier.

31. A combination product as claimed in claim 30 which comprises a pharmaceutical formulation including a compound as defined in claim 1, but without the proviso, or a pharmaceutically-acceptable salt thereof, another therapeutic agent that is useful in the treatment of inflammation, and a pharmaceutically-acceptable adjuvant, diluent or carrier.

32. A combination product as claimed in claim 30 which comprises a kit of parts comprising components:
(a) a pharmaceutical formulation including a compound as defined in claim 1, but without the proviso, or a pharmaceutically-acceptable salt thereof, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
which components (a) and (b) are each provided in a form that is suitable for administration in conjunction with the other.

33. A process for the preparation of a compound as defined in claim 1, which comprises:
(i) for compounds of formula I in which R¹ and R² represent the same optionally substituted aryl or heteroaryl group, reaction of a compound of formula II, 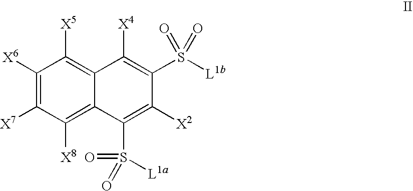
wherein L^{1a} and L^{1b} independently represent a suitable leaving group and X², X⁴, X⁵ to X⁸ are as defined in claim 1, with a compound of formula III,
R^{x}—NH₂ III
wherein R^{x} represents both R¹ and/or R² (as appropriate);
(ii) reaction of a compound of formula IV, 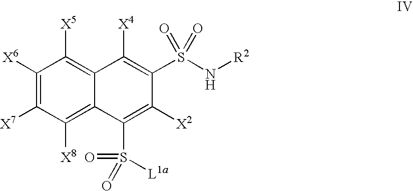
or a compound of formula V, 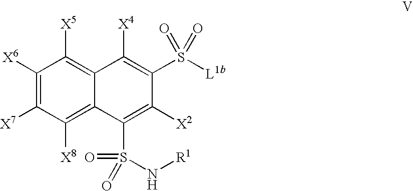
wherein X², X⁴, X⁵ to X⁸, R¹ and R² are as defined in claim 1 and L^{1a} and L^{1b} are as defined above, with a compound of formula III as defined above;
(iii) for compounds of formula I in which X⁸ represents Z², in which Z² represents halo, —R^{3a}, —C(O)R^{3b}, —C(O)OR^{3c}, —C(O)N(R^{4a})R^{5a}, —S(O)ₘR^{3j} or —S(O)₂N(R^{4h})R^{5h}, and R^{3b}, R^{4a}, R^{5a}, R^{4h} and R^{5h} are as defined in claim 1, provided that they do not represent hydrogen, and R^{3a}, R^{3c} and R^{3j} are as defined in claim 1, reaction of a compound corresponding to a compound of formula I but in which X⁸ represents a metal (e.g. lithium), with a compound of formula VI,
Z^{x}-L² VI
wherein L² represents a suitable leaving group, such as chloro, bromo or iodo and Z^{x} represents halo, —R^{3a}, —C(O)R^{3b}, —C(O)OR^{3c}, —C(O)N(R^{4a})R^{5a}, —S(O)ₘR^{3j} or —S(O)₂N(R^{4h})R^{5h}, and R^{3b}, R^{4a}, R^{5a}, R^{4h} and R^{5h} are as defined in claim 1, provided that they do not represent hydrogen, and R^{3a}, R^{3c} and R^{3j} are as defined in claim 1;
(iv) for compounds of formula I in which a substituent Z¹ or Z² is present and represents —N(R^{4b})R^{5b} in which R^{5b} is H and R^{4b} is as defined in claim 1, hydrolysis of a corresponding compound of formula I in which the relevant substituent is —N(R^{4b})C(O)OR^{4c} in which R^{4b} and R^{4c} are as defined in claim 1, or a protected derivative thereof;
(v) for compounds of formula I in which a substituent Z¹ or Z² is present and represents —C(O)OR^{3c} and/or —OC(O)OR^{3p} and R^{3c} and R^{3p} are as defined in claim 1, trans-esterification of a corresponding compound of formula I in which R^{3c} and R^{3p} do not represent the same value as the value of R^{3c} and R^{3p} in the compound of formula I to be prepared;
(vi) for compounds of formula I in which a substituent Z¹ or Z² is present and represents —C(O)OR^{3c}, —C(O)N(R^{4a})R^{5a}, —N(R^{4b})R^{5b}, —N(R^{3e})C(O)N(R^{4d})R^{5d}, —N(R^{3f})C(O)OR^{4e}, —N(R^{3g})S(O)₂N(R^{4f})R^{5f}, —OR^{3h}, —OC(O)N(R^{4g})R^{5g}, —OC(O)OR^{3p} and/or —S(O)₂N(R^{4h})R^{5h} and R^{3e}, R^{3f}, R^{3g}, R^{3h}, R^{4a}, R^{4b}, R^{4d}, R^{4e}, R^{4f}, R^{4g}, R^{4h}, R^{5a}, R^{5b}, R^{5d}, R^{5f}, R^{5g} and R^{5h} are as defined in claim 1, provided that they do not represent hydrogen, and R^{3c} and R^{3p} are as defined in claim 1, reaction of a compound corresponding to a compound of formula I in which R^{3c} and/or R^{3p} represents hydrogen or a corresponding compound of formula I in which R^{3e}, R^{3f}, R^{3g}, R^{3h}, R^{4a}, R^{4b}, R^{4d}, R^{4e}, R^{4f}, R^{4g}, R^{4h}, R^{5a}, R^{5b}, R^{5d}, R^{5f}, R^{5g} and/or R^{5h} represent hydrogen (as appropriate), or an appropriate anion thereof, with a compound of formula VII,
R^{3a}-L³ VII
wherein L³ represents a suitable leaving group and R^{3a} is as defined in claim 1;
(vii) for compounds of formula I in which a substituent Z¹ or Z² is present and represents halo, —CN, —N(R^{4b})R^{5b}, —N(R^{3d})C(O)R^{4c}, —N(R^{3e})C(O)N(R^{4d})R^{5d}, —N(R³)C(O)OR^{4e}, —N(R^{3g})S(O)₂N(R^{4f})R^{5f}, —OR^{3h} and/or —N(R^{3k})S(O)₂R^{3m}, and R^{3d}, R^{3e}, R^{3f}, R^{3g}, R^{3h}, R^{3k}, R^{3m}, R^{4b}, R^{4c}, R^{4d}, R^{4e}, R^{4f}, R^{5b}, R^{5d} and R^{5f} are as defined in claim 1, reaction of a corresponding compound of formula I in which Z¹ or Z² (as appropriate) represents a suitable leaving group, with a halogen, or an appropriate reagent that is a source of a halogen, or with a compound of formula VIII,
Z^{y}-H VIII
wherein Z^{y} represents —CN, —N(R^{4b})R^{5b}, —N(R^{3d})C(O)R^{4c}, —N(R^{3e})C(O)N(R^{4d})R^{5d}, —N(R^{3f})C(O)OR^{4e}, —N(R^{3g})S(O)₂N(R^{4f})R^{5f}, —OR^{3h} or —N(R^{3k})S(O)₂R^{3m}, and R^{3d}, R^{3e}, R^{3f}, R^{3g}, R^{3h}, R^{3k}, R^{3m}, R^{4b}, R^{4c}, R^{4d}, R^{4e}, R^{4f}, R^{5b}, R^{5d} and R^{5f} are as defined in claim 1, or a suitable derivative thereof.

34. A process for the preparation of a pharmaceutical formulation as defined in claim 21, which process comprises bringing into association a compound of formula I, as defined in claim 1 but without the proviso, or a pharmaceutically acceptable salt thereof with a pharmaceutically-acceptable adjuvant, diluent or carrier.

35. A process for the preparation of a combination product as defined in claim 30, which process comprises bringing into association a compound of formula I, as defined in claim 1 but without the proviso, or a pharmaceutically acceptable salt thereof with another therapeutic agent that is useful in the treatment of inflammation, and a pharmaceutically-acceptable adjuvant, diluent or carrier.
